SEQUENCE LISTING (5 sequences)

SEQ ID NO: 1 - protein, 695 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 2 - protein, 697 aa, Mus musculus
MRLTVGALLACAALGLCLAVPDKTVKWCAVSEHENTKCISFRDHMKTVLPPDGPRLACVKKTSYPDCIKAISASEADAMTLDGGWVYDAGLTPNNLKPVAAEFYGSVEHPQTYYYAVAVVKKGTDFQLNQLEGKKSCHTGLGRSAGWVIPIGLLFCKLSEPRSPLEKAVSSFFSGSCVPCADPVAFPKLCQLCPGCGCSSTQPFFGYVGAFKCLKDGGGDVAFVKHTTIFEVLPEKADRDQYELLCLDNTRKPVDQYEDCYLARIPSHAVVARKNNGKEDLIWEILKVAQEHFGKGKSKDFQLFSSPLGKDLLFKDSAFGLLRVPPRMDYRLYLGHNYVTAIRNQQEGVCPEGSIDNSPVKWCALSHLERTKCDEWSIISEGKIECESAETTEDCIEKIVNGEADAMTLDGGHAYIAGQCGLVPVMAEYYESSNCAIPSQQGIFPKGYYAVAVVKASDTSITWNNLKGKKSCHTGVDRTAGWNIPMGMLYNRINHCKFDEFFSQGCAPGYEKNSTLCDLCIGPLKCAPNNKEEYNGYTGAFRCLVEKGDVAFVKHQTVLDNTEGKNPAEWAKNLKQEDFELLCPDGTRKPVKDFASCHLAQAPNHVVVSRKEKAARVKAVLTSQETLFGGSDCTGNFCLFKSTTKDLLFRDDTKCFVKLPEGTTPEKYLGAEYMQSVGNMRKCSTSRLLEACTFHKH

SEQ ID NO: 3 - protein, 698 aa, Rattus norvegicus
MRFAVGALLACAALGLCLAVPDKTVKWCAVSEHENTKCISFRDHMKTVLPADGPRLACVKKTSYQDCIKAISGGEADAITLDGGWVYDAGLTPNNLKPVAAEFYGSLEHPQTHYLAVAVVKKGTDFQLNQLQGKKSCHTGLGRSAGWIIPIGLLFCNLPEPRKPLEKAVASFFSGSCVPCADPVAFPQLCQLCPGCGCSPTQPFFGYVGAFKCLRDGGGDVAFVKHTTIFEVLPQKADRDQYELLCLDNTRKPVDQYEDCYLARIPSHAVVARNGDGKEDLIWEILKVAQEHFGKGKSKDFQLFGSPLGKDLLFKDSAFGLLRVPPRMDYRLYLGHSYVTAIRNQREGVCPEGSIDSAPVKWCALSHQERAKCDEWSVSSNGQIECESAESTEDCIDKIVNGEADAMSLDGGHAYIAGQCGLVPVMAENYDISSCTNPQSDVFPKGYYAVAVVKASDSSINWNNLKGKKSCHTGVDRTAGWNIPMGLLFSRINHCKFDEFFSQGCAPGYKKNSTLCDLCIGPAKCAPNNREGYNGYTGAFQCLVEKGDVAFVKHQTVLENTNGKNTAAWAKDLKQEDFQLLCPDGTKKPVTEFATCHLAQAPNHVVVSRKEKAARVSTVLTAQKDLFWKGDKDCTGNFCLFRSSTKDLLFRDDTKCLTKLPEGTTYEEYLGAEYLQAVGNIRKCSTSRLLEACTFHKS

SEQ ID NO: 4 - protein, 703 aa, Equus caballus
MRLAIRALLACAVLGLCLAEQTVRWCTVSNHEVSKCASFRDSMKSIVPAPPLVACVKRTSYLECIKAIADNEADAVTLDAGLVFEAGLSPYNLKPVVAEFYGSKTEPQTHYYAVAVVKKNSNFQLNQLQGKKSCHTGLGRSAGWNIPIGLLYWQLPEPRESLQKAVSNFFAGSCVPCADRTAVPNLCQLCVGKGTDKCACSNHEPYFGYSGAFKCLADGAGDVAFVKHSTVLENLPQEADRDEYQLLCRDNTRKSVDEYKDCYLASIPSHAVVARSVDGKEDLIWGLLNQAQEHFGTEKSKDFHLFSSPHGKDLLFKDSALGFLRIPPAMDTWLYLGYEYVTAIRNLREDIRPEVPKDECKKVKWCAIGHHEKVKCDEWSVNSGGNIECESAQSTEDCIAKIVKGEADAMSLDGGFIYIAGKCGLVPVLAENYETRSGSACVDTPEEGYHAVAVVKSSSDPDLTWNSLKGKKSCHTGVDRTAGWNIPMGLEIKHCEFDKFFREGCAPGYRRNSTLCNLCIGSASGPGRECEPNNHERYYGYTGAFRCLVEKGDVAFVKHQTVEQNTDGRNPDDWAKDLKSENFKLLCPDGTRKSVTEFKSCYLARAPNHAVVSRKEKAACVCQELHNQQASYGKNGSHCPDKFCLFQSATKDLLFRDDTQCLANLQPTTTYKTYLGEKYLTAVANLRQCSTSRLLEACTFHRV

SEQ ID NO: 5 - protein, 695 aa, Bos taurus
MRPAVRALLACAVLGLCLADPERTVRWCTISTHEANKCASFRENVLRSGPFVSCVKKTSHMDCIKAISNNEADAVTLDGGLVYEAGLKPNNLKPVVAEFHGTKDNPQTHYYAVAVVKKDTDFKLNELRGKKSCHTGLGRSAGWNIPMGKLYKELPDPQESIQRAAANFFSASCVPCADQSSFPKLCQLCAGKGTDKCACSNHEPYFGYSGAFKCLMEGAGDVAFVKHSTVFDNLPNPEDRKNYELLCGDNTRKSVDDYQECYLAMVPSHAVVARTVGGKEDVIWELLNHAQEHFGKDKPDNFQLFQSPHGKDLLFKDSADGFLKIPSKMDFELYLGYEYVTALQNLRESKPPDSSKDECMVKWCAIGHQERTKCDRWSGFSGGAIECETAENTEECIAKIMKGEADAMSLDGGYLYIAGKCGLVPVLAENYKTEGESCKNTPEKGYLAVAVVKTSDANINWNNLKDKKSCHTAVDRTAGWNIPMGLKINNCKFDEFFSAGCAPGSPRNSSLCALCIGSEKGTGKECVPNSNERYYGYTGAFRCLVEKGDVAFVKDQTVIQNTDGNNNEAWAKNLKKENFEVLCKDGTRKPVTDAENCHLPNHAVVSRKDKATCVEKILNKQQDDFGKSVTDCTSNFCLFQSNSKDLLFRDDTKCLASIAKKTYDSYLGDDYVRAMTNLRQCSTSKLLEACTFHKP

(19) United States
(12) Patent Application Publication
(10) Pub. No.: US 2009/0162425 A1
(43) Pub. Date: Jun. 25, 2009

(54) METHODS AND COMPOSITIONS FOR INHIBITING UNDESIRABLE CELLULAR PROLIFERATION BY TARGETED LIPOSOME DELIVERY OF ACTIVE AGENTS

(75) Inventors: Murali Krishna Divi, Ronkonkoma, NY (US); George C. Wood, Bartlett, TN (US); M. Waleed Gaber, Germantown, TN (US); Laura Thoma, Memphis, TN (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: University of Tennessee Research Foundation, Knoxville, TN (US)

(21) Appl. No.: 12/234,282

(22) Filed: Sep. 19, 2008

Related U.S. Application Data
(60) Provisional application No. 60/994,463, filed on Sep. 19, 2007.

(51) Int. Cl. A61K 9/127; A61K 31/337; A61P 35/04

(52) U.S. Cl. 424/450; 514/449

(57) ABSTRACT

Provided are methods for inhibiting undesirable proliferation of a tumor cell or a tumor in a subject including administering to the subject a composition that includes an effective amount of an active agent, wherein the active agent is entrapped by one or more liposomes, the active agent has activity in inhibiting undesirable proliferation of the tumor cell or the tumor, and the one or more liposomes include one or more targeting agents that preferentially or specifically bind to a binding molecule expressed by the tumor cell or the tumor, present on the tumor cell or the tumor, present in the tumor cell or the tumor, or combinations thereof. Also provided are compositions for treating tumors, particularly gliomas, in a subject in need thereof.

### CROSS-REFERENCE TO RELATED APPLICATION

The presently disclosed subject matter claims the benefit of U.S. Provisional Patent Application Ser. No. 60/994,463, filed Sep. 19, 2007, the disclosure of which is incorporated herein by reference in its entirety.

### TECHNICAL FIELD

The presently disclosed subject matter relates to compositions and methods for inhibiting undesirable cellular proliferation. In some embodiments, the compositions and methods of the presently disclosed subject matter relate to targeted liposome-based anti-tumor therapy. The presently disclosed subject matter also relates to liposomes comprising active agents and having outer surfaces that include binding molecules that target the liposome to tumors and tumor cells. In some embodiments, the binding molecules comprise transferrin polypeptides, or fragments or derivatives thereof, which bind to transferrin receptors present on tumor cells or in tumors. In some embodiments, the presently disclosed subject matter provides for the treatment of transferrin-expressing tumors and/or tumor cells in subjects in need thereof by providing liposomes loaded with paclitaxel and having outer surfaces comprising transferrin polypeptides that target tumors and/or tumor cells.

### BACKGROUND

Primary brain tumors are a common cause of cancer related deaths. High-grade gliomas are the most common type of primary brain tumor, and affected patients have a median survival of less than 1 year. Most malignant gliomas are incurable with presently available treatments. Currently accepted therapeutic adjuvants to surgery, such as radiotherapy and chemotherapy, provide only a minor improvement in the disease course and life expectancy for patients diagnosed with malignant glioma. Chemotherapy often fails to substantially improve the prognosis of malignant glioma because of significant local and systemic toxicity, problems with transport of the drug across the blood brain barrier (BBB), and high degrees of chemoresistance demonstrated by tumor cells (e.g., glioma cells).

Accordingly, there is a long felt and continuing need in the art for effective brain tumor treatments that address these obstacles.

TABLE OF ABBREVIATIONS

| | |
|---|---|
| BBB | Blood brain barrier |
| CCD | Charge coupled device |
| CED | Convection enhanced delivery |
| DSPC | 1,2-Distearoyl-sn-glycero-3-phosphocholine |
| DSPE-PEG | 1,2-Distearoyl-sn-glycero-3-phosphoethanolamine- |
| | N-[PEG(2000)] conjugate |
| EPC | Egg phosphatidylcholine |
| GFP | Green fluorescent protein |
| HPGFC | High performance gel filtration chromatography |
| HPLC | High performance liquid chromatography |
| Hr | Hour |
| HSPC | hydrogenated soybean phosphatidylcholine |
| kDa | kilodalton |
| LCL | Long-circulating liposomes (Non-targeted liposomes) |
| LUVs | Large unilamellar vesicles |
| MAb | Monoclonal antibody |
| MLVs | Multilamellar vesicles |
| MPS | Mononuclear phagocytic system |
| MWCO | Molecular weight cutoff |
| PBS | Phosphate buffered saline |
| PDI | Polydispersity index |
| PEG | Polyethylene glycol |
| PL | Phospholipid |
| PSD | Particle size distribution |
| RES | Reticular endothelial system |
| SD | Standard deviation |
| SSTL | Sterically stabilized targeted liposomes |
| SUVs | Small unilamellar vesicles |
| T_{1/2} | Circulation half-life |
| Tf-LCL | Transferrin-coupled long-circulating liposomes |
| | (targeted liposomes) |
| T_{g} | Glass transition temperature |
| Tₘ | Phase transition temperature |

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods for inhibiting undesirable proliferation of a tumor cell or a tumor in a subject. In some embodiments, the methods comprise administering to the subject a composition comprising an effective amount of an active agent, wherein (i) the active agent is entrapped by one or more liposomes; (ii) the active agent has activity in inhibiting undesirable proliferation of the tumor cell or the tumor; and (iii) the one or more liposomes comprise one or more targeting agents that preferentially or specifically bind to a binding molecule expressed by the tumor cell or the tumor, present on the tumor cell or the tumor, present in the tumor cell or the tumor, or combinations thereof, whereby the liposomes deliver the active agent to the tumor cell or the tumor. In some embodiments, the tumor cell or the tumor is a glioma cell or a glioma. In some embodiments, one or more of the one or more liposomes are long-circulating liposomes. In some embodiments, the long-circulating liposomes comprise about 1-7% of DSPE-PEG2000 in total lipids. In some embodiments, the one or more liposomes are less than about 300 nm in diameter. In some embodiments, the one or more liposomes are about 100 nm in diameter. In some embodiments, the active agent is selected from the group consisting of docetaxel, camptothecin, carmustine/BCNU, lomustine/CCNU, vincristine, vinblastine, doxorubicin, and paclitaxel. In some embodiments, the active agent is paclitaxel. In some embodiments, the active agent is contained within an interior space of the one or more liposomes, attached to the one or more liposomes, present within a bilayer of the one or more liposomes, or a combination thereof. In some embodiments, the one or more targeting agents comprises a transferrin polypeptide or a fragment or derivative thereof that preferentially or specifically binds to a binding molecule present on or in the tumor cell or the tumor. In some embodiments, the binding molecule comprises a transferrin receptor that specifically binds to the transferrin polypeptide or the fragment or derivative thereof. In some embodiments, the transferrin polypeptide is expressed at substantially greater levels on or in the tumor cell or the tumor than in normal tissue surrounding the tumor cell or the tumor in the subject. In some embodiments, the one or more liposomes are suspended in a pharmaceutically acceptable carrier. In some embodiments, the administering comprises intravenously injecting the composition into the subject. In some embodiments, the administering comprising delivering the composition intracranially to the subject. In some embodiments, the inhibiting comprises reducing the size of the tumor, reducing tumor load, reducing tumor growth rate, or a combination thereof in the subject.

The presently disclosed subject matter also provides targeted liposome delivery systems for treating a tumor cell or a tumor. In some embodiments, the targeted liposome delivery systems comprise (i) a plurality of liposomes, each liposome comprising an interior space, a lipid bilayer, and an exterior surface; (ii) an active agent present within the interior space of the liposome, present within the lipid bilayer of the liposome, attached to the exterior surface of the liposome, or a combination thereof; and (iii) a targeting agent that preferentially or specifically binds to a binding molecule present on or in the tumor cell or the tumor attached to the liposome. In some embodiments, the plurality of liposomes are lyophilized for enhancement of long-term stability. In some embodiments, the plurality of liposomes are produced by a lipid hydration method followed by extrusion. In some embodiments, the liposome comprises one or more lipids selected from the group consisting of egg phosphatidylcholine (EPC), hydrogenated soybean phosphatidylcholine (HSPC), cholesterol, 1,2-distearoyl-glycero-3-phosphoethanolamine-N-[PEG(2000)] conjugate (DSPE-PEG), DSPE-PEG-Maleimide, and DSPE-PEG-biotin. In some embodiments, the targeting agent is attached to the exterior surface of the liposome through an avidin-biotin bond or a thioether bond. In some embodiments, the plurality of liposomes are suspended in a pharmaceutically acceptable carrier. In some embodiments, the active agent is selected from the group consisting of docetaxel, camptothecin, carmustine/BCNU, lomustine/CCNU, vincristine, vinblastine, doxorubicin, and paclitaxel. In some embodiments, the active agent is paclitaxel. In some embodiments, the targeting agent comprises a transferrin polypeptide or a fragment or derivative thereof, and the binding molecule comprises a transferrin receptor that preferentially or specifically binds to the transferrin polypeptide or the fragment or derivative thereof.

The presently disclosed subject matter also provides anti-tumor therapeutic compositions for treating a tumor comprising a suspension of liposomes in a pharmaceutically acceptable carrier. In some embodiments, the liposomes comprise (a) an interior space, a lipid bilayer, and an exterior surface; (b) an active agent contained within the interior space of the liposome, present within the lipid bilayer of the liposome, attached to the exterior surface of the liposome, or a combination thereof; and (c) one or more transferrin polypeptides or fragments or derivatives thereof attached to the liposome. In some embodiments, the active agent is selected from the group consisting of docetaxel, camptothecin, carmustine/BCNU, lomustine/CCNU, vincristine, vinblastine, doxorubicin, and paclitaxel. In some embodiments, the active agent is paclitaxel.

Thus, it is an object of the presently disclosed subject matter to provide methods for inhibiting undesirable proliferation of a glioma cell or of a glioma in a subject.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIG. 1 is a photograph depicting a transmission electron micrograph of the transferrin conjugated long circulating liposomes. Magnification: 250,000×. The scale bar in the lower right corner is 100 nm.

FIG. 2 is a photograph depicting a transmission electron micrograph of the non-conjugated long circulating liposomes. Magnification: 250,000×. The scale bar in the lower right corner is 100 nm.

FIG. 3 is a graph showing blood clearance of 1% PEG liposomes. Sprague Dawley rats received fluorescent-labeled formulations via intravenous injection and the serum fluorescence concentrations were measured at different time intervals. Each value is the mean of 3 independent experiments. ♦: control solution lacking liposomes; ▪: 80 nm 1% liposomes.

FIG. 4 is a graph showing blood clearance of liposomes made with different concentrations of PEG lipid. Sprague Dawley rats received fluorescent labeled formulations via intravenous injection. Serum fluorescence concentrations were measured at different time intervals. Each value is the mean of 3 independent experiments. ♦: 80 nm 5% PEG liposomes; ▪: 80 nm 1% PEG liposomes; ▴: 80 nm 0% PEG liposomes.

FIG. 5 is a graph showing blood clearance of different size liposomes in Sprague Dawley rats. The rats were administered intravenously fluorescent-labeled formulations and the serum fluorescence concentrations were measured at different time intervals. Each value is the mean of 3 independent experiments. ♦: 80 nm 5% PEG liposomes; ▪: 300 nm 5% PEG liposomes; ▴: 3000 nm 5% PEG liposomes.

FIG. 6 is a graph showing blood clearance of non-targeted and targeted liposomes. Sprague Dawley rats received fluorescent-labeled formulations via intravenous injection. Serum fluorescence concentrations were measured at different time intervals. Each value is the mean of 3 independent experiments. ♦: transferrin-conjugated liposomes; ▪: liposomes lacking transferrin conjugation.

FIGS. 7A-7I are a series of intravital fluorescence images of brain microvasculature.

FIG. 7A is a background image with white light (1.3×).

FIG. 7B is a background image with white light (10×).

FIG. 7C is a background image with 1,1′,di-octadecyl-3,3,3′3′-tetramethylindocarbocyanine perchlorate (Dil) filter (555 nm Excitation/575 nm Emission).

FIG. 7D is an image of 0 hours after Tf-LCL injection.

FIG. 7E is an image of 1 hour after Tf-LCL injection.

FIG. 7F is an image of 4 hours after Tf-LCL injection.

FIG. 7G is an image of 6 hours after Tf-LCL injection.

FIG. 7H is an image of 24 hours after Tf-LCL injection.

FIG. 7I is an image of 48 hours after Tf-LCL injection.

FIG. 8 is a series of whole body near infrared images of mice showing tumor localization of control solution, long circulating liposomes, and transferrin receptor targeted liposomes. Column 1 (i.e., the left most column) shows three white light images showing the location of the tumors. Column 2 shows images taken with a GFP filter showing the C6 GFP tumor area. Column 3 shows background images with a DIR filter before injection. Columns 4-8 show images taken after injection of DIR labeled formulations at the indicated time points.

FIG. 9 is a graph showing localization of DIR labeled formulations in glioma tumors. The pixel intensity of DIR fluorescence signal of formulations in glioma tumor tissue was quantified. Transferrin conjugated liposomes showed faster and higher targeting ability to tumor tissue when compared to long circulating liposomes and solution formulations. ♦: negative control solution; ▪: Tf-LCL; Δ: LCL.

FIG. 10 is a bar graph showing selective accumulation of Tf-LCL in C6 GFP glioma tumors in mice. High fluorescence signals in tumor tissue were observed up to 48 hours. The fluorescence ratio between tumor and normal muscle tissue yielded up to 10-fold selectivity for the tumor in comparison with surrounding normal tissue; n=3; Mean±SD. Tumor targeting index: 10.59±1.08.

FIG. 11 is a series of whole body near infrared images of mice. Tumor localization of different size long circulating liposomes was shown. Column 1 (i.e., the left most column) shows white light image showing the location of the tumors. Column 2 shows images with a GFP filter showing the C6 GFP tumor area. Column 3 shows background images with a DIR filter before injection. Columns 4-8 show images taken after injection of DIR labeled formulations at the indicated time points.

FIG. 12 is a series of photomicrographs showing C6 GFP flank tumor sections from tumor targeting study. Tumors show the presence of DIR fluorescence with 100 nm long circulating liposomes and transferrin-conjugated long circulating liposomes.

FIG. 13 is a bar graph showing the size and size distribution of the paclitaxel long circulating liposomes. A dynamic light scattering method was used. Average diameter: 132.8 nm. PDI: 0.167.

FIG. 14 is a bar graph showing the size and size distribution of transferrin-conjugated long circulating liposomes using dynamic light scattering method. Average diameter: 141.3 nm. PDI: 0.170.

FIG. 15 is a graph showing a C6 GFP tumor growth curve. Tumor volume was determined after repeated caliper measurements from 2 to 18 days after tumor cell inoculation. Group mean values (±S.D.) for these mice are shown.

FIG. 16 is a series of photographs of nude mice showing the presence of tumors in the flank regions of each mouse. The top row shows an increase in tumor area with time using a GFP filter. The bottom row shows the respective white light images at corresponding time points.

FIG. 17 is a graph showing a C6 GFP tumor growth curve. Tumor area was determined after repeated tumor imaging from 2 to 18 days after tumor cell inoculation. Group mean values (±S.D.) for these mice are shown.

FIG. 18 is a graph showing tumor growth of subcutaneously inoculated C6 GFP tumor cells. The length and width of each tumor were measured using calipers, and mean tumor volumes were calculated for each group. Tumor growth was significantly delayed after treatment with paclitaxel-entrapped liposomes compared to solution and no treatment controls. Δ: paclitaxel in Tf-LCL; □: paclitaxel in LCL; ⋄: paclitaxel in solution; *: no treatment control. P<0.05 for Tf-LCL vs. all other groups; n=4-5 per group; mean±SD.

FIG. 19 is a series of near infrared fluorescence images of brains isolated from mice. Selective localization of Tf-LCL in intracranial GFP expressing C6 glioma brain tumors was observed. The top row images were taken with white light, the second row images were taken with a GFP filter, and the third row images were taken with a DIR filter.

FIG. 20 is a bar graph showing selective accumulation of Tf-LCL in intracranial glioma tumors. High fluorescence signals in mouse intracranial tumor tissue were observed up to 24 hours. The fluorescence ratio between tumor and normal muscle tissue yielded up to 6-fold selectivity for the tumor in comparison with surrounding normal tissue; n=3; Mean±SD. Tumor targeting index: 6.15±3.3.

FIG. 21 is a bar graph showing in vivo efficacy of paclitaxel against C6 intracranial glioma tumors. C6 GFP glioma cells were inoculated into the frontal lobe of the brains of nude mice (n=3-5 per group). After 5 days of tumor cell incubation, groups were treated by retroorbital injection every 24 hours for 9 subsequent days with 2 mg/kg of paclitaxel in Tf-LCL or LCL. Paclitaxel in Tf-LCL was efficacious at reducing the tumor burden (p=0.038). However, no significant tumor reduction was observed with paclitaxel in LCL (p=0.188).

FIGS. 22A-22C are images of C6 GFP intracranial glioma tumors. Animals treated with paclitaxel in Tf-LCL (FIG. 22B) as compared to the no treatment control (FIG. 22A) and paclitaxel in LCL (FIG. 22C) are shown. Top row shows reference dots for GFP filter that were created with an FITC-containing solution.

FIGS. 23A and 23B are graphs of particle size distribution (PSD) of targeted liposomes after lyophilization.

FIG. 23A is a graph of the original PSD before freeze-drying. Average size: 137 nm. Polydispersity index (PDI): 0.176.

FIG. 23B is a graph of the PSD after freeze-drying a formulation containing 15% (w/v) extra-liposomal sucrose. Average size: 150 nm. PDI: 0.215.

FIGS. 24A and 24B are graphs showing the effects of sucrose and trehalose on liposome size after lyophilization.

FIG. 24A is a graph showing PSD after lyophilizing the formulation containing 15% (w/v) extra-liposomal sucrose. Average size: 150 nm. PDI: 0.215.

FIG. 24B is a graph showing PSD after lyophilizing the formulation containing 15% (w/v) extra-liposomal trehalose. Average size: 160 nm. PDI: 0.277.

FIGS. 25 A-25C are graphs showing a lyoprotective effect of sucrose and trehalose.

FIG. 25A is a graph of the original zeta potential before freeze-drying. Average zeta potential: −17.6 mV.

FIG. 25B is a graph of zeta potential after freeze-drying with 15% (w/v) extra-liposomal trehalose. Average zeta potential: −21.3 mV.

FIG. 25C is a graph of zeta potential after freeze-drying with 15% (w/v) extra-liposomal sucrose. Average zeta potential: −20.4 mV.

FIG. 26 is an alignment of transferrin amino acid sequences from five species: Human, Mouse, Rat, Horse, and Bovine using the ClustalIX program (Thompson et al., 1997). The GENBANK® Accession Nos. that correspond to these amino acid sequences are given in Table 1. In FIG. 26, the degree of homology at each amino acid position among the five species is indicated by the height of the histograms below each group of 80 amino acids and also above each group (*—amino acids are conserved in all five species; :—amino acids are highly homologous in all five species; .—amino acids are somewhat homologous in all five species; no symbol: amino acids are divergent in and/or missing in one or more species).

### DETAILED DESCRIPTION

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a cell” includes a plurality of cells (e.g., a tissue and/or an organ), “a liposome” includes a plurality of liposomes, and so forth.

The term “about”, as used herein when referring to a measurable value such as an amount of weight, time, etc. is meant to encompass variations of, in some embodiments, ±20% or ±10%, in some embodiments, ±5%, in some embodiments, ±1%, in some embodiments, ±0.5%, and in some embodiments, ±0.1%, from the specified amount, as such variations are appropriate to perform the disclosed methods.

As used herein, “treatment” means any manner in which one or more of the symptoms of a disorder are ameliorated or otherwise beneficially altered. Thus, the terms “treating” or “treatment” of a disorder as used herein includes: reverting the disorder (e.g., causing regression of the disorder or its clinical symptoms wholly or partially); preventing the disorder (e.g., causing the clinical symptoms of the disorder not to develop in a subject that can be exposed to or predisposed to the disorder but does not yet experience or display symptoms of the disorder); inhibiting the disorder (e.g., arresting or reducing the development of the disorder or its clinical symptoms); attenuating the disorder (e.g., weakening or reducing the severity or duration of a disorder or its clinical symptoms); or relieving the disorder (e.g., causing regression of the disorder or its clinical symptoms). Further, amelioration of the symptoms of a particular disorder by administration of a particular composition can include any lessening, whether permanent or temporary, lasting or transient that can be attributed to or associated with administration of a composition of the presently disclosed subject matter and/or practice of the presently disclosed methods.

In some embodiments, of the presently disclosed subject matter, treatment can include treating a tumor. In some embodiments, the tumor size and/or tumor burden can be substantially reduced upon treatment of the tumor. In some embodiments, tumor growth can be substantially delayed upon treatment of the tumor.

The term “administering” as used herein refers to a method of delivering or providing a composition or compound to a desired site, tissue, organ, system or subject. In some embodiments, the “administering” comprises parenteral administration, including injection and/or infusion methods. In some embodiments, the “administering” comprises intravenous injection, intravenous infusion, and/or a retro orbital route of administration.

The terms “effective amount” and “therapeutically effective amount” as used herein refer to any amount of an active agent sufficient to produce a measurable and/or desirable response (e.g., a biologically or clinically relevant response in a subject being treated) in a tissue, system, animal or human that is being sought by a researcher, veterinarian, medical doctor or other clinician. Actual dosage levels of active ingredients in the compositions of the presently disclosed subject matter can be varied so as to administer an amount of the active compound(s) that is effective to achieve the desired therapeutic response for a particular subject. The selected dosage level can depend upon the activity of the therapeutic composition, the route of administration, combination with other drugs or treatments, the severity of the condition being treated, and/or the condition and/or prior medical history of the subject being treated. By way of example and not limitation, doses of compositions can be started at levels lower than that believed to be required to achieve the desired therapeutic effect, and gradually increased until the desired effect is achieved. The potency of a composition can vary, and therefore an “effective amount” can vary. In some embodiments, the “effective amount” can refer to the amount of active agent that is sufficient for treat a tumor or tumor tissues in a subject. In some embodiments, the “effective amount” can refer to the amount of anti-tumor active agent that is sufficient to reduce the size and/or growth rate of a tumor, in some embodiments a glioma, in a subject.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, gender, severity and stage of symptoms, and the presence of additional deleterious physical conditions.

As used herein, the phrase “active agent” refers to a molecule or combination of molecules that when in contact with a cell inhibits the proliferation of that cell. As would be recognized by one of ordinary skill in the art, an active agent can act in a cell independent method, cell autonomously, or both, and can do so either be remaining outside of the target cell or before, during, and/or after entering the target cell. Thus, in some embodiments an active agent directly inhibits proliferation of a tumor cell, and in some embodiments indirectly inhibits proliferation of a tumor cell (e.g., by disrupting its vascular network). Exemplary active agents include, but are not limited to therapeutic agents (e.g., chemotherapeutic agents, radiotherapeutic agents, etc.). In some embodiments, an active agent comprises a lipophilic chemotherapeutic agent, which in some embodiments can be selected from the group consisting of docetaxel, camptothecin, carmustine/BCNU, lomustine/CCNU, vincristine, vinblastine, doxorubicin, and paclitaxel. In some embodiments, the active agent is paclitaxel.

As used herein, the phrase “active agent” also refers to a molecule or combination of molecules that when in contact with a cell or tissue allows the cell or tissue to be imaged and/or visualized. As would be recognized by one of ordinary skill in the art, an active agent in this context can do so either by binding to a molecule present on the surface of the target cell and/or by entering the target cell. Exemplary imaging agents include, but are not limited to, paramagnetic, radioactive, and fluorogenic ions. In some embodiments, an imaging agent comprises a radioactive imaging agent. Exemplary radioactive imaging agents include, but are not limited to, gamma-emitters, positron-emitters, and x-ray-emitters. Particular radioactive imaging agents include, but are not limited to, ⁴³K, ⁵²Fe, ⁵⁷Co, ⁶⁷Cu, ⁶⁷Ga, ⁶⁸Ga, ⁷⁷Br, ⁸¹Rb/^{81MM}Kr, ^{87m}Sr, ^{99m}Tc, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb, and ²⁰⁶Bi. Other radioactive and non-radioactive imaging agents known to one skilled in the art can also be employed in the compositions and methods of the presently disclosed subject matter.

The term “pharmaceutically acceptable” as used herein refers to a material that is not biologically or otherwise undesirable, i.e., the material can be incorporated into a pharmaceutical composition administered to a subject without causing any undesirable biological effects or interacting in a deleterious manner with any of the other components of the composition in which it is contained. When the term “pharmaceutically acceptable” is used to refer to a pharmaceutical carrier, it is implied that the carrier has met the required standards of toxicological and manufacturing testing or that it is included on the Inactive Ingredient Guide prepared by the U.S. Food and Drug Administration. In some embodiments, “pharmaceutically acceptable” refers to a material that is pharmaceutically acceptable for use in humans.

In some embodiments, the compositions employed in the compositions and methods of the presently disclosed subject matter can take such forms as suspensions, solutions, or emulsions in oily or aqueous vehicles, and can contain formulatory agents such as suspending, stabilizing, and/or dispersing agents. By way of example and not limitation, compositions of the presently disclosed subject matter can comprise liposomes suspended in water containing 8.3% CREMOPHOR® EL and 8.2% ethanol.

The term “subject” as used herein refers to any invertebrate or vertebrate species. The methods disclosed herein are particularly useful in the treatment of warm-blooded vertebrates. Thus, the presently disclosed subject matter concerns mammals and birds. More particularly, provided is the treatment of mammals such as humans, as well as those mammals of importance due to being endangered (such as Siberian tigers), of economic importance (animals raised on farms for consumption by humans), and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, provided is the treatment of livestock, including, but not limited to, domesticated swine (pigs and hogs), ruminants, horses, poultry, and the like.

The term “tumor” as used herein can refer to any abnormal growth or mass of tissue in a subject. A “tumor” can be either malignant or benign. In some embodiments, a “tumor” can be a tumor in the head, neck, brain, or central nervous system of a subject. In some embodiments, the “tumor” can be a glioma. In some embodiments, the “tumor” can be present in a subject. In some embodiments, the tumor is present in a mammalian subject (e.g., a human subject).

The term “receptor” as used herein refers to a protein that binds to a specific molecule or ligand. In some embodiments, the “receptor” can be located on the membrane or surface of a cell or tissue. In some embodiments, the “receptor” can be expressed by a tumor, such as a glioma. By way of example and not limitation, a “receptor” of the presently disclosed subject matter can be a transferrin receptor, or a fragment or derivative thereof that preferentially or specifically binds to transferrin or a fragment or derivative thereof.

Transferrin receptor is a representative target for receptor-mediated targeting of tumors and tumor cells (e.g., gliomas and glioma cells). The density of transferrin receptor on glioma cells is correlated with the extent of cell growth and division. Neoplastic cells (e.g., tumor cells) ordinarily divide faster than normal cells and consequently express more transferrin receptors than their surroundings. This discrepancy is even more appreciable in the stable (i.e., quiescent) environment of the brain. The extent and diffuseness of transferrin receptor expression in glioma cells have been shown to be significantly greater than in normal brain tissue, with expression linked to the severity of the tumor. The relative over-expression of these transferrin receptors offers the prospect for favorable targeting of brain tumors relative to surrounding normal tissue.

The term “ligand” as used herein refers to a molecule (e.g., a ““targeting agent” as defined herein) that is able to bind to and form a complex with a receptor (e.g., a “binding molecule” as defined herein). A “ligand” can bind to a receptor at a binding site on the receptor by intermolecular forces such as ionic bonds, hydrogen bonds, and/or Van der Waals forces. By way of example and not limitation, a “ligand” of the presently disclosed subject matter can comprise a transferrin polypeptide or a fragment or derivative thereof that is capable of preferential or specific binding to a transferrin receptor including, but not limited to the transferrin polypeptides that correspond to the GENBANK® Accession Nos. listed in Table 1.

The term “liposome” as used herein refers to a (typically) spherical vesicle comprising a lipid bilayer and having an interior space. In some embodiments, a liposome can comprise one or more lipids selected from egg phosphatidylcholine (EPC), hydrogenated soybean phosphatidylcholine (HSPC), cholesterol, 1,2-distearoyl-glycero-3-phosphoethanolamine-N-[PEG(2000)] conjugate (DSPE-PEG), DSPE-PEG-Maleimide, DSPE-PEG-biotin, and combinations thereof. In some embodiments, a liposome is a long circulating liposome. In some embodiments, a long circulating liposome comprises one or more surface modifications, which can result in decreased recognition and subsequent phagocytosis of the liposomes by cells of the mononuclear phagocytic system (MPS). In some embodiments, a long circulating liposome comprises one or more surface modifications with hydrophilic polymers such as polyethylene glycol (PEG), which can result in decreased recognition and subsequent phagocytosis of the liposomes by cells of the mononuclear phagocytic system (MPS).

Liposomes can be versatile carriers for targeted drug delivery by the intravenous route. In some embodiments, liposomes can be produced by a lipid hydration method followed by an extrusion method.

In some embodiments, the liposomes are conjugated to one or more transferrin polypeptides or fragments or derivates thereof via a linker or tether. Any linker or tether than can be employed to conjugate one or more transferrin polypeptides or fragments or derivates thereof to a liposome can be employed, exemplary embodiments of which are known to one of ordinary skill in the art and include, but are not limited to chemical linkers, polyethylene glycol linkers, and avidin or streptavidin/biotin technology. In some embodiments, one or more transferrin polypeptides or fragments or derivates thereof are conjugated to liposomes using a streptavidin-biotin bond, wherein the liposome comprises a biotin moiety and the one or more transferrin polypeptides or fragments or derivates thereof comprise an avidin moiety.

In addition to comprising a transferrin polypeptide or a fragment or derivative thereof, the liposomes of the presently disclosed subject matter further comprise an entrapped active agent. As used herein, the term “entrap”, and grammatical variants thereof, refers to an association between the liposome and the active agent wherein delivery of the liposome to a target cell or a target tissue also delivers the active agent to the target cell or the target tissue (e.g., a tumor cell or a tumor). Entrapped active agents can be associated with liposomes in various manners including, but not limited to being encapsulated within an interior space of the liposome, being attached to an outer surface of a liposome, being present within the lipid bilayer of the liposome, or any combination thereof, all of which are intended to be encompassed by the term “entrapped”. In some embodiments, the liposomes entrap one or more active agents selected from the group consisting of docetaxel, camptothecin, carmustine/BCNU, lomustine/CCNU, vincristine, vinblastine, doxorubicin, and paclitaxel.

### II. Targeted Liposomal Delivery Systems

II.A. General Design Considerations

Development of effective targeted drug delivery systems can include consideration of many design and scientific aspects. In general, design characteristics that can be considered include:
1) Choice of target organ for selection of passive or active targeting method;
2) Selection of a targeting antigen or receptor that has homogeneous over expression and has a vital role in tumor progression with minimal expression in normal tissue;
3) Selection of a stable, specific, and non-immunogenic targeting ligand that can undergo endocytosis efficiently;
4) A stable liposome formulation with small (in some embodiments less than 100 nm) size, and zeta potential of in some embodiments ˜20 mV;
5) Minimal leakage of drug;
6) Long in vivo circulating properties;
7) Minimal interaction with blood components;
8) Selective extravasation into tumors along with high tumor localization capability;
9) A stable and rapid conjugation method with high conjugation efficiency; and
10) A highly potent anticancer drug with a possibility for high liposome encapsulation.

Each of these design considerations as well as their interactions can play a role in determining the success of a targeted liposome delivery system.

Based on the target organ, the liposomal targeting method can be selected. By regulating the liposome properties like size, charge, and composition, liposomes can be designed to deliver their contents to specific sites such as liver, spleen, and permeable vasculature of some solid tumors by passive targeting. However, active targeting can be a preferred option to target pathological sites like brain tumors that have limited accessibility.

For efficient targeting, the targeted receptor or antigen can have homogeneous, high expression on the surface of the target cells. The antigen or receptor should preferably not be down regulated. Circulating shed antigen can compete with the target cells for binding of the targeted therapeutics, and any complexes that form might be cleared from the circulation. The receptors or antigens that undergo endocytosis of antibodies can increase efficacy of drug delivery, presumably by inducing tumor cells to endocytose immunoliposomes (Allen, 2002).

Several factors can also determine the selection of a targeting ligand. Non-antibody ligands are relatively stable, can be inexpensive to manufacture, and can be readily available. However, they can bind to some non-target tissues. Endogenous ligands like folic acid and transferrin found in significant levels in body fluids and the free ligands can compete for binding with the targeted therapy (Allen, 2002). For a high degree of selectivity towards target tissues, monoclonal antibodies or antibody fragments can be selected. Cost, stability, and potential immunogenicity are major disadvantages with antibodies.

Along with the choice of a ligand, the ligand density can have a potential impact on targeting efficiency of a targeted liposome. High ligand densities are preferable for enhancing the binding to target cells. However, high ligand density can lead to rapid clearance from the circulation. Additionally, achieving high ligand density can be expensive and difficult to achieve (Pardridge, 1999a; Pardridge, 1999b; Pardridge, 1999c).

II.B. Liposome Formulations

Delivery of drugs to brain tumors using targeted liposomes can involve numerous complications. Exemplary difficulties associated with targeted delivery systems that need to be overcome include rapid clearance from the circulation and minimal interaction of targeted delivery system with target cells after reaching the target site. Tumor targeted liposomes minimally should exhibit accumulation properties comparable to non-targeted liposomes.

Several physical factors play key roles in inhibiting liposome targeting following extravasation. Factors such liposome composition, size, charge, and the nature of the vascular barrier, tumor structure, and binding site can affect the efficacy of targeted liposome. Beyond the targeting strategy, several formulation issues need to be addressed for development of stable targeted liposomal delivery systems. The production of liposomes, with attached targeting ligands, can involve an extensive number of modification steps. Manufacturing and quality factors like batch-to-batch reproducibility, entrapment efficiency, particle size control, and scale up are exemplary problems limiting the manufacturing and development of commercially viable targeted liposomal dosage forms.

As such, the presently disclosed subject matter provides in some embodiments targeted liposome delivery systems for treating a tumor cell or a tumor. In some embodiments of the presently disclosed subject matter, the targeted liposome delivery systems comprise a plurality of liposomes, each liposome comprising an interior space, a lipid bilayer, and an exterior surface. The presently disclosed subject matter also provides anti-tumor therapeutic compositions for treating a tumor, wherein the anti-tumor therapeutic compositions comprise a suspension of the presently disclosed liposomes in a pharmaceutically acceptable carrier. In some embodiments, a liposome formulation for targeted drug delivery of the presently disclosed subject matter is stable with small (less than about 100 nm) size, zeta potential of about −15 to about −25 mV, minimal leakage of drug, long in vivo circulating properties, minimal interaction with blood components, selective extravasation into tumors, and high tumor localization capability.

II.B.1. Composition

Liposomes comprising saturated phospholipids such as HSPC and DSPC along with optimum levels of cholesterol can produce stable liposomes with minimal leakage. The fluidity of liposome bilayers can be altered by using phospholipids with different Tₘ, which in turn can vary depending upon the length and nature (saturated or unsaturated) of the fatty acid chains. Liposomes containing high phase transition temperature lipids (Tₘ>37° C.) can be rigid at the physiological temperature and can be less leaky. In contrast, liposomes composed of low Tₘ lipids (Tₘ<37° C.) can be more susceptible to leakage of drugs encapsulated in aqueous phase at physiological temperatures. The fluidity of bilayers also influences interaction of liposomes with plasma components and cell membranes. Liposomes composed of high Tₘ lipids were reported to have lower extent of uptake by RES, compared to those containing low Tₘ lipids (Drummond et al., 1999). Incorporation of cholesterol into the lipid bilayer increases membrane rigidity thereby affecting their stability both in vitro and in vivo. For hydrophobic drugs, unsaturated lipids like egg phosphatidylcholine or egg sphingomyelin can enhance drug loading into liposomes. Anti-oxidants like α-tocopherol in the formulation can reduce auto-oxidation of lipid components and prolong the shelf lives of liposomes.

II.B.2. Liposome Size

The in vivo circulation half life and tumor accumulation of liposomes can depend on the sizes of the liposomes. Small unilamellar liposomes (e.g., ≦100 nm) are generally opsonized less rapidly and to a lower extent than large liposomes (e.g., >300 nm), and therefore the rate liposome uptake by the reticular endothelial system (RES) generally increases with the size of the vesicle (Harashima et al., 1994; Papisov, 1998). Inclusion of PEG-lipids in the liposome composition can result in clearance rates that are relatively insensitive to size in the range of 80 to 250 nm.

Optimal liposome size can depend on the tumor target. The macromolecular size of liposomes typically prevents them from passing through the 2 nm pores found in the endothelium of blood vessels in most healthy tissues as well as the 6 nm pores found in post capillary venules. In tumor tissue, the vasculature is generally discontinuous, and pore sizes can vary from less than 100 to 780 nm or more, which can allow for accumulation of liposomes in these areas (Yuan et al., 1995).

II.B.3. Liposome Surface Charge

Surface charge properties of liposomal colloidal systems can influence their drug carrier potentials, since such properties can impact the interactions of liposomes with plasma proteins, and the lipid composition can influence the liposomal surface charge. Lack of a surface charge can reduce the physical stability of small unilamellar liposomes by increasing their aggregation. However, negatively charged liposomes have been believed to be more rapidly removed from circulation than neutral or positively charged liposomes. Negatively charged liposomes can be taken up by cells through coated-pit endocytosis, while cationic liposomes can deliver their contents to cells either by fusion with cell membranes or through coated pit endocytosis.

II.B.4. Steric Stabilization

Colloidal stability of liposomes can be enhanced by addition of hydrophilic polymers or glycolipids like PEG conjugated lipids, ganglioside-GM1, or phosphatidylinositol into liposomes. Sterically stabilized liposomes can show prolonged lifetimes in the circulation as compared with non-stabilized liposomes. Sterically stabilized liposomes can also be less reactive toward serum proteins and less susceptible to RES uptake than non-stabilized liposomes. PEG can induces dehydration of the head group region of lipids because PEG, when chemically attached to a lipid head group, undergoes steric exclusion from the liposome surface in a similar way to free PEG. This can result in greater density of the grafted PEG further from the surface. Thus, the local concentration gradient of PEG chains from the liposome surface leads to an osmotic imbalance, changes in thermodynamic properties, and hydration of the lipids. At higher concentrations (e.g., >10 mol %) PEG chains can be in a highly overlapped brush configuration. Due to repulsion of PEG chains, weakening of the bilayer packing has been observed at these high concentrations of PEG lipids. Although sterically stabilized liposomes can be characterized by prolonged circulation time and decreased liposomal uptake by the RES, they do not function themselves to actively target the liposome to tumors (Tirosh et al., 1998).

II.B.5. Lyophilization of Targeted Liposomes

The therapeutic application of targeted liposomes can be dependent on the physical integrity and stability of the lipid bilayer structure. In the liquid state, liposome formulations are subject to both physical and chemical instability. These stability parameters can be important to the in vivo behavior of liposomal drug delivery systems. Liposomal size distribution can be a relevant parameter with respect to the pharmacokinetic and pharmacodynamic behavior of drugs that are site-specifically targeted in vivo.

One of the practical challenges with liposomes for delivery of drugs to target cells is that liposomes are relatively unstable during storage. Lyophilization is a potential method for enhancement of long-term stability of liposomes. In the lyophilization process, most of the water molecules are excluded from a specimen and the aqueous suspension becomes a powder that can be stored at selected, even at ambient, temperatures. Prior to use, reconstitution of the particulate system can be achieved by rehydration of the dry powder. Additionally, removal of water by lyophilization prevents hydrolysis of phospholipids. Other chemical and physical degradation processes can also be retarded by low molecular mobility in the solid phase. Further, freeze-drying of liposome formulations, if performed successfully, can result in a pharmaceutically useful dry cake that can be reconstituted within seconds to obtain the original dispersion.

Lyophilization of targeted liposomes can be more complex when compared to large multilamellar conventional liposomes. Liposome bilayer membranes can be damaged during the lyophilization cycle by mechanical stress caused by the high pressures that vesicle membranes can be exposed to during ice crystal formation and chemically from increased concentrations of solute during freezing and dehydration. This can lead to undesirable aggregation and fusion of the vesicles as well as leakage of the entrapped compounds upon reconstitution of the lyophilized cake. In the absence of cryoprotectants, small targeted liposomes can be converted in to large multilamellar liposomes upon lyophilization and subsequent reconstitution (Peer et al., 2003). This change in size of the liposomes can be detrimental for targeted drug delivery. Cryoprotectants have been shown to decrease vesicle fusion and leakage caused by both freeze-thaw and the freeze-drying process (Sun et al., 1996; Crowe et al., 1997; Crowe et al., 1998; Van Winden & Crommelin, 1999). Sugars including trehalose, sucrose, mannose, and glucose have been used as cryoprotectants at high concentrations (e.g., about 30%) in liposome preparations. Among these sugars, trehalose is particularly effective in preserving liposomes. Crowe et al. have described possible mechanisms by which sugars protect biological membranes during freeze-drying (Crowe et al., 1997; Crowe et al., 1998). Cryoprotectants are non-eutectic in nature, forming an amorphous frozen matrix upon cooling. The freeze process generally occurs very quickly in the presence of cryoprotectants upon cooling to the freezing point depression. The role attributed to these cryoprotectants is replacement of structure-stabilizing water-based hydrogen bonds at the liposomal surface, which are lost in the process of drying (Van Winden & Crommelin, 1999).

Several factors can affect the stability of the liposomes in the dry state (Crowe & Crowe, 1988). These factors include, but are not limited to size and charge of liposomes, type and concentration of stabilizing sugar, and the dry mass ratio between the stabilizing sugar and lipid. Small (e.g., smaller than about 60 nm) and large (e.g., larger than about 1000 nm) lyophilized liposomes are found to be unstable even in extremely high concentrations of sugars (Crowe & Crowe, 1988). The stability of lyophilized liposomes can be significantly increased by the addition of small amount of negatively charged lipid in the bilayer. The efficacy of sugar depends on the size of liposomes and dry mass ratio between the stabilizing sugar and lipid (Crowe & Crowe, 1988). Cryoprotection of liposomes is typically greatest when formulating with these sugars at high concentrations (5% to 20%; Jovanovic et al., 2006). Vesicle fusion can be decreased at lower concentrations than are needed to minimize leakage. Prevention of leakage is generally maximized when the sugar is present both inside and outside the liposome (Crowe & Crowe, 1988). Advantages of formulating liposomes with trehalose include being less reactive than reducing sugars, higher Tg′ than sucrose, a high melting temperature (100° C. at 2% moisture), low hygroscopicity, and FDA approval as an injectable ingredient (Crowe et al., 1987; Crowe, 2007).

Peer et al. have reported lyophilization of targeted unilamellar liposomes without added sugars. Hyaluronan, a surface bound ligand in certain targeted bioadhesive liposomes, also protected liposomes during freeze-drying process. Peer et al. proposed that hyaluronan, like sugars, protects liposomes by providing substitute structure-stabilizing hydrogen bonds (Peer et al., 2003). Ugwu et al. have reported lyophilization of liposome formulations of mitoxantrone using sucrose as a cryoprotectant (Ugwu et al., 2005). Sucrose was found to be more effective in minimizing drug leakage from the lyophilized liposomes. Long-term stability studies showed that lyophilized formulation was stable for up to 13 months when stored at refrigerated condition. 5-fluorouracil lyophilized liposomes with saccharose as a cryoprotectant have also been reported, in which saccharose cryoprotection yielded a stable and less permeable 5-fluorouracil lyophilized liposome formulation (Glavas-Dodov et al., 2005). Cui et al. also reported a novel lyophilized liposome system with tertiary butyl alcohol/water co-solvent system (Cui et al., 2006). This process gave a stable free flowing lyophilized powder with sucrose as a cryoprotectant.

The above subsections thus provide guidance that one of ordinary skill in the art can employ in the preparation and use of the methods and compositions of the presently disclosed subject matter.

II.C. Targeting Moieties

In some embodiments, a targeted liposome comprises a targeting ligand that targets the liposome to a desired site of accumulation.

The term “targeting”, as used herein to describe the in vivo activity of a ligand following administration to a subject, refers to the preferential movement and/or accumulation of a ligand (also referred to herein as a “binding molecule”) in a target tissue as compared with a control tissue.

The term “selective targeting” as used herein refers to a preferential localization of a ligand that results in an amount of ligand in a target tissue that is in some embodiments about 2-fold greater than an amount of ligand in a control tissue, in some embodiments an amount that is about 5-fold or greater, and in some embodiments an amount that is about 10-fold or greater. The terms “targeting agent” and “targeting molecule” as used herein each refer to a ligand that displays targeting activity. In some embodiments, a targeting ligand displays selective targeting.

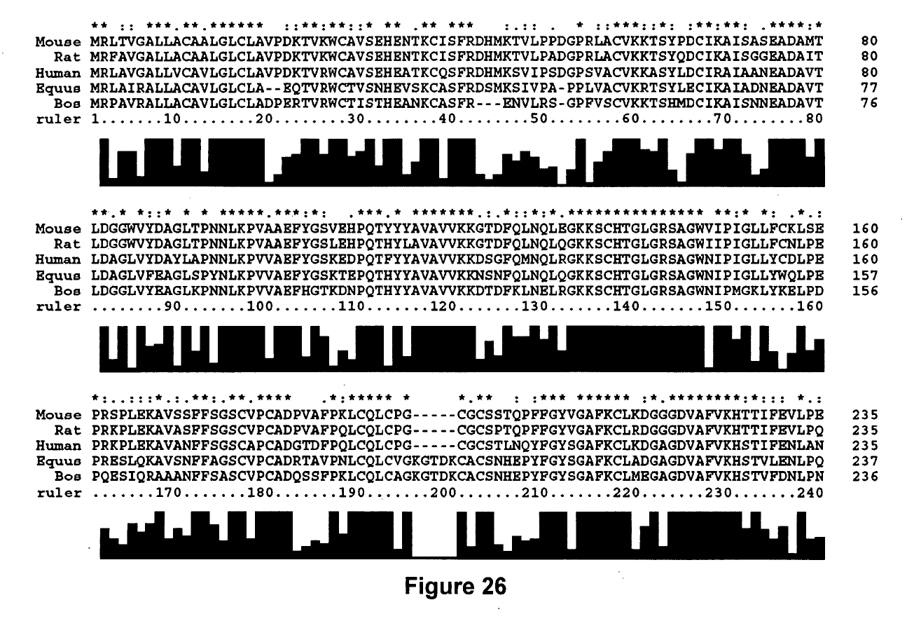

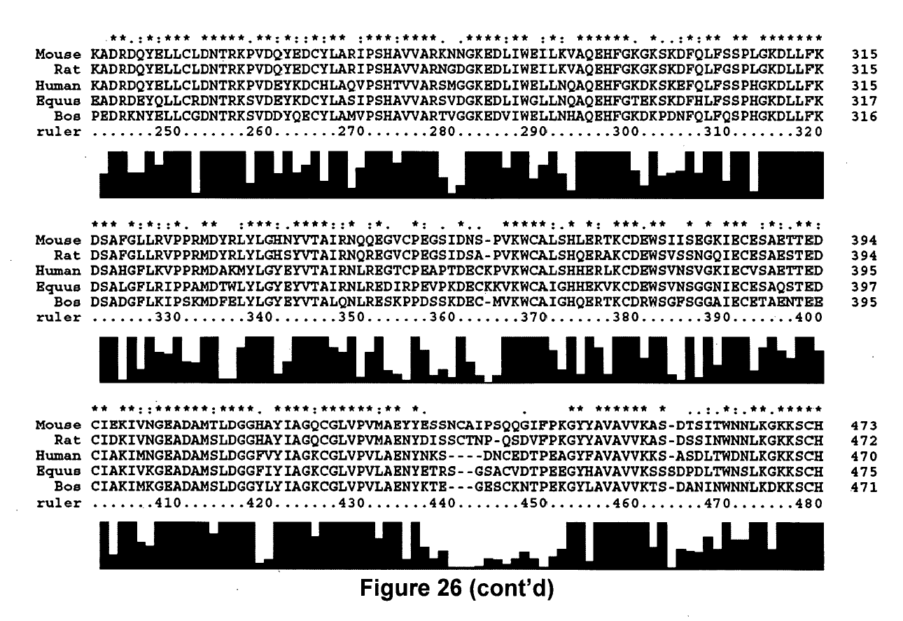

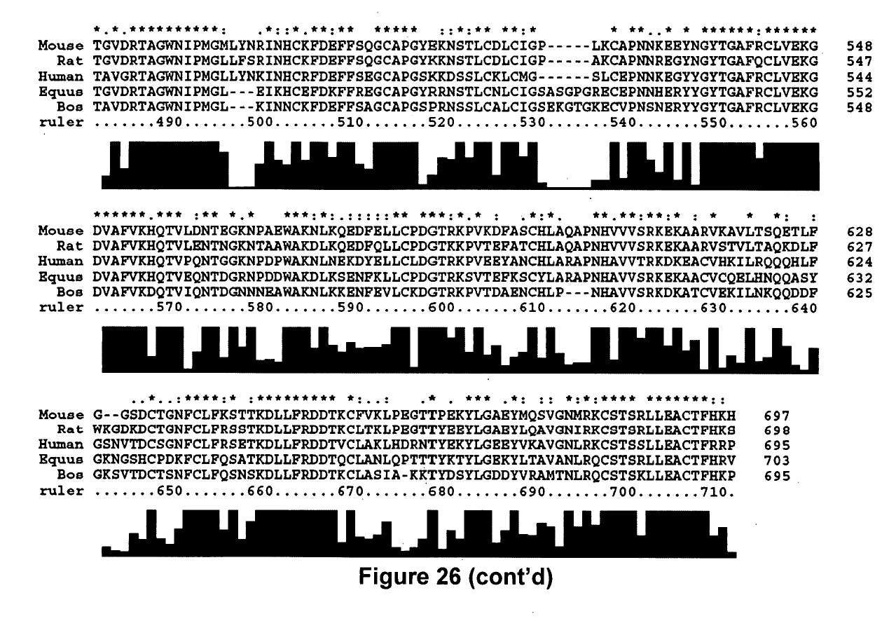

The term “targeting agent” thus refers to a molecule that can bind to a target cell or a target tissue to preferentially or specifically target the target cell or the target tissue by binding to a binding molecule expressed on or in the target tissue and/or a cell thereof. In some embodiments, targeting agents that bind to target cells and/or target tissues display substantially no binding (e.g., no binding or only background binding) to non-target cells and tissues. In some embodiments, a targeting agent preferentially or specifically binds to a target molecule present on the surface of a target cell or tissue. In some embodiments, a targeting agent comprises a transferrin polypeptide or a fragment or derivative thereof, and the binding molecule comprises a transferrin receptor or a functional fragment or derivative thereof. In some embodiments, the transferrin is a human transferrin or a functional fragment or derivative thereof). GENBANK® Accession Numbers for nucleotide and amino acid sequences of transferring from humans and other exemplary non-limiting species are presented in Table 1. An alignment of the amino acid sequences in Table 1 is presented in FIG. 26. GENBANK® Accession Numbers for nucleotide and amino acid sequences of transferrin receptors from humans and other exemplary non-limiting species are presented in Table 2.

TABLE 1
GENBANK ® Accession Nos. for Exemplary Transferrins

| Species | Nucleotide Sequence | Amino Acid Sequence |
|---|---|---|
| Homo sapiens | NM_001063 | NP_001054; |
| | | SEQ ID NO: 1 |
| Mus musculus | NM_133977 | NP_598738; |
| | | SEQ ID NO: 2 |
| Rattus norvegicus | NM_001013110 | NP_001013128; |
| | | SEQ ID NO: 3 |
| Equus caballus | NM_001081946 | NP_001075415; |
| | | SEQ ID NO: 4 |
| Bos taurus | NM_177484 | NP_803450; |
| | | SEQ ID NO: 5 |

TABLE 2
GENBANK ® Accession Nos. for
Exemplary Transferrin Receptors

| | Nucleotide Sequence | |
|---|---|---|
| Species | (ORF) | Amino Acid Sequence |
| Homo sapiens | NM_003234 | NP_003225 |
| | (284-2566) | |
| Mus musculus | NM_011638 | NP_035768 |
| | (50-2341) | |
| Rattus norvegicus | XM_340999 | XP_341000 |
| | (137-2419) | |
| Equus caballus | NM_001081913 | NP_001075382 |
| | (1-2304) | |
| Canis lupus familiaris | NM_001003111 | NP_001003111 |
| | (1-2313) | |
| Sus scrofa | NM_214001 | NP_999166 |
| | (36-2342) | |

The term “binding” refers to an affinity between two molecules, for example, a ligand and a target molecule. As used herein, “binding” refers to a preferential binding of one molecule for another in a mixture of molecules. The binding of a ligand to a target molecule can be considered specific if the binding affinity is about 1×10⁴ M⁻¹ to about 1×10⁶ M⁻¹ or greater.

The phrases “specifically binds” and “selectively binds”, when referring to the binding capacity of a ligand, refer to a binding reaction that is determinative of the presence of the protein in a heterogeneous population of proteins and other biological materials. The phrase “specifically binds” also refers to selectively targeting, as defined herein above.

The phases “substantially lack binding” or “substantially no binding”, as used herein to describe binding of a ligand in a control tissue, refers to a level of binding that encompasses non-specific or background binding, but does not include specific binding.

As used herein, the phrase “transferrin or a fragment or derivative thereof” refers to a member of the TF gene family exemplified by the biosequences set forth in Table 1 and in the Sequence Listing. Thus, in some embodiments a transferrin polypeptide comprises an amino acid sequence as set forth in any of GENBANK® Accession Nos. NP_{—}001054, NP_{—}598738, NP_{—}001013128, NP_{—}001075415, and NP_{—}803450, as well as all known orthologs thereof.

With respect to fragments of a transferrin polypeptide, the presently disclosed subject matter is intended to encompass any polypeptide that comprises a subsequence of a transferrin polypeptide amino acid sequence sufficient to allow it to selectively or preferentially binding to a transferrin receptor (including but not limited to those transferrin receptors that correspond to the GENBANK® Accession Nos. presented in Table 2) present in the subject of interest.

Similarly, a derivative of a transferrin polypeptide is a modified transferrin polypeptide that includes one or more deletions, insertions, and/or amino acid substitutions relative to a naturally occurring transferrin, provided that the derivative is capable of selectively or preferentially binding to a transferrin receptor (including but not limited to those transferrin receptors that correspond to the GENBANK® Accession Nos. presented in Table 2) present in the subject of interest.

In some embodiments, a modified transferrin polypeptide includes one or more amino acid substitutions relative to a transferrin naturally occurring in the species to which the subject belongs, with the proviso that whatever modifications the modified transferrin might have do not prevent the modified transferrin from binding to a transferrin receptor that is present in the subject. Methods for generating modified transferrin polypeptides and assaying what effect, if any, the modifications have on binding of the modified transferrin to a transferrin receptor would be understood by those of ordinary skill in the art after consideration of the instant disclosure.

The twenty conventional amino acids and their abbreviations follow conventional usage. See Golub & Gren, 1991, incorporated herein by reference in its entirety. In some embodiments, a modified transferrin polypeptide comprises one or more modified amino acids. As used herein, the phrase “modified amino acids” refers to an amino acid that is not one of the conventional twenty amino acids. Modified amino acids include, but are not limited to stereoisomers (e.g., D-amino acids) of the twenty conventional amino acids, unnatural amino acids such as α-, α-disubstituted amino acids, N-alkyl amino acids, lactic acid, and other unconventional amino acids. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

In some embodiments, a modified transferrin polypeptide comprises one or more conservative amino acid substitutions relative to a naturally occurring transferrin. Naturally occurring residues can be divided into classes based on common side chain properties: hydrophobic (norleucine (Nor), Met, Ala, Val, Leu, Ile); neutral hydrophilic (Cys, Ser, Thr, Asn, Gln); acidic (Asp, Glu); basic (His, Lys, Arg); residues that influence chain orientation (Gly, Pro); and aromatic (Trp, Tyr, Phe). See Creighton, 1983; Henikoff & Henikoff, 2000. Conservative amino acid substitutions can involve exchange of a member of one of these classes with another member of the same class. Conservative amino acid substitutions can also encompass non-naturally occurring amino acid residues, which are typically incorporated by chemical peptide synthesis rather than by synthesis in biological systems. These include peptidomimetics and other reversed or inverted forms of amino acid moieties.

In some embodiments, one of ordinary skill in the art can consider the amino acid sequences of orthologous transferring (i.e., the primary sequences of transferrin polypeptides from species other than the species to which the subject belongs) in determining how to modify a transferrin polypeptide without disrupting its ability to bind to a transferrin receptor in a subject. FIG. 26 shows an amino acid alignment among transferrins from five different species, and the information provided therein can be informative as the skilled artisan considers amino acids that would be more or less likely to be mutable without negatively impacting the ability of a modified transferrin to bind to a transferrin receptor in vivo. For example, amino acid positions that are identical among the five transferrin polypeptides depicted in FIG. 26 might be, although will not necessarily be, less preferred candidates for substitution than other amino acid positions where the primary sequences of the depicted transferrin polypeptides show more divergence.

In some embodiments, the hydropathic index of amino acids can be considered in making amino acid substitutions. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see e.g., Kyte et al., 1982). It is known that certain amino acids can be substituted for other amino acids having a similar hydropathic index or score and still retain a similar biological activity. In some embodiments, amino acids with hydropathic indices that are within ±2 are substituted when making changes based upon the hydropathic index. In some embodiments, those that are within ±1 are substituted, and in some embodiments, those within ±0.5 are substituted.

It is also understood in the art that the substitution of like amino acids can be made effectively on the basis of hydrophilicity. As detailed in U.S. Pat. No. 4,554,101, the following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In some embodiments, amino acids with hydrophilicity values that are within ±2 are substituted when making changes based upon the hydrophilicity value. In some embodiments, those that are within ±1 are substituted, and in some embodiments, those within ±0.5 are substituted.

Amino acid codes, including codons encoding each amino acid, are set forth in Table 3. Exemplary conservative amino acid substitutions are set forth in Table 4.

TABLE 3
Amino Acid Abbreviations, Codes, and
Functionally Equivalent Codons

| Amino Acid | 3-Letter | 1 -Letter | Codons |
|---|---|---|---|
| Alanine | Ala | A | GCA; GCC; GCG; GCU |
| Arginine | Arg | R | AGA; AGG; CGA; CGC; |
| | | | CGG; CGU |
| Asparagine | Asn | N | AAC; AAU |
| Aspartic Acid | Asp | D | GAC; GAU |
| Cysteine | Cys | C | UGC; UGU |
| Glutamic acid | Glu | E | GAA; GAG |
| Glutamine | Gln | Q | CAA; CAG |
| Glycine | Gly | G | GGA; GGC; GGG; GGU |
| Histidine | His | H | CAC; CAU |
| Isoleucine | Ile | I | AUA; AUC; AUU |
| Leucine | Leu | L | UUA; UUG; CUA; CUC; |
| | | | CUG; CUU |
| Lysine | Lys | K | AAA; AAG |
| Methionine | Met | M | AUG |
| Phenylalanine | Phe | F | UUC; UUU |
| Proline | Pro | P | CCA; CCC; CCG; CCU |
| Serine | Ser | S | ACG; AGU; UCA; UCC; |
| | | | UCG; UCU |
| Threonine | Thr | T | ACA; ACC; ACG; ACU |
| Tryptophan | Trp | W | UGG |
| Tyrosine | Tyr | Y | UAC; UAU |
| Valine | Val | V | GUA; GUC; GUG; GUU |

TABLE 4
Amino Acid Substitutions

| | Exemplary | Primary |
|---|---|---|
| Original | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Ile |
| | (Nor) | |
| Leu | Nor, Ile, Val, Met, Ala, Phe | Ile |
| Lys | Arg, 1,4-diaminobutyric acid, Gln, Asn | Arg |
| Met | Leu, Phe, Ile | Leu |
| Phe | Leu, Val, Ile, Ala, Tyr | Leu |
| Pro | Ala, Gly | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Nor | Leu |

Transferrin receptor (TfR) currently shows promise as a target molecule for receptor-mediated targeting of glioma. The density of TfR is correlated with the extent of cell growth and division. Neoplastic cells ordinarily divide faster than normal cells and consequently express more TfR than their surroundings. This discrepancy is even more appreciable in the stable environment of the brain. The extent and diffuseness of TfR expression in glioma has been shown to be significantly greater than in normal brain tissue, with expression linked to the severity of tumor. The relative over expression of these transferrin receptors offers the potential for favorable targeting of brain tumors over its surrounding normal tissue. Transferrin has also be widely applied as a targeting ligand in the active targeting of anticancer agents, proteins, and genes to primary proliferating malignant cells that overexpress transferrin receptors.

II.D. Choice of Active Agents

For optimal targeted liposomal delivery, the active agent should be compatible with the liposome structure and should show efficient loading into the liposomes. The leakage rate of the active from the liposomes can limit the choice of active agents that are available for liposomal targeted delivery. Highly lipophilic drugs, for example, tend to associate mainly with the bilayer compartment of the liposome. This can result in lower entrapment stability due to faster redistribution of the drug to plasma components (Sharma & Sharma, 1997). Even though amphipathic active agents are reported to be suitable for liposomal carriers, a targeted liposomal system can benefit from optimization for each active agent (Drummond et al., 1999).

Paclitaxel (trade name TAXOL®) is a diterpenoid pseudoalkaloid isolated from Taxus brevifolia, discovered at the Research Triangle Institute (RTI; Research Triangle Park, North Carolina, United States of America) in 1967. Paclitaxel is approved by the FDA for the treatment of ovarian and breast cancers. Paclitaxel was the first of a new class of microtubule stabilizing agents and is considered as one of the most significant advances in chemotherapy of the past 15-20 years.

Paclitaxel promotes the polymerization of tubulin. Specifically, paclitaxel binds to the β subunit of tubulin. Tubulin is the “building block” of microtubules, and the binding of paclitaxel locks these building blocks in place. The tubulin/paclitaxel complex is extraordinarily stable and does not disassemble. This causes cell death by disrupting the normal tubule dynamics required for cell division (Sharma & Straubinger, 1994; Hennenfent & Govindan, 2006; Slavin et al., 2007). Paclitaxel is also known to induce programmed cell death (apoptosis) in cancer cells by binding to an apoptosis stopping protein called Bcl-2 (B-cell leukemia 2), thus arresting its function (Henley et al., 2007). Normal cells are also affected by exposure to paclitaxel, but cancer cells are far more susceptible to paclitaxel treatment.

Paclitaxel has anti-neoplastic activity particularly against primary epithelial ovarian carcinoma, breast cancer, colon, head, non-small cell lung cancer, and AIDS-related Kaposi's sarcoma (Rowinsky & Donehower, 1993; Rowinsky et al., 1993; Markman et al., 1994; Rowinsky et al., 1994). Paclitaxel is also used for the prevention of restenosis of coronary stents (Sun & Eikelboom, 2007).

Paclitaxel has a molecular weight of 853 Daltons (Da). It is a white to off-white crystalline powder, is highly lipophilic, is insoluble in water, and has a melting point of 216-217° C. It is characterized by biphasic plasma clearance. The generally accepted dose is 200-250 mg m⁻² and is given as 3 and 24 hour infusions. Terminal half-life was found to be in the range of 1.3-8.6 hours (mean 5 hours; Rowinsky & Donehower, 1993; Rowinsky et al., 1994). The drug undergoes an extensive P450 mediated hepatic metabolism, and less than 10% of the drug in the unchanged form is excreted in the urine. More than 90% of the drug binds rapidly and extensively to plasma proteins (Rowinsky et al., 1993).

Paclitaxel is poorly soluble in an aqueous medium, but can be dissolved in organic solvents. An accepted value of aqueous solubility of paclitaxel is 0.6 mM (Tarr & Yalkowsky, 1987). The solubility of paclitaxel is not affected by changes in pH due to lack of functional groups that are ionizable in the pharmaceutically acceptable pH range (Tarr & Yalkowsky 1987).

Paclitaxel is currently formulated in a vehicle composed of 1:1 blend of CREMOPHOR® EL (BASF Corp., Florham Park, N.J., United States of America) and ethanol, which is diluted 5-20-fold in normal saline or dextrose solution (5%) for administration. This formulation is stable in unopened vials for 5 years at 4° C. Several attempts have been made to develop an aqueous-based formulation of paclitaxel, including co-solvency, micellar solubilization, emulsification, cyclodextrins, nanoparticle and liposome formations, that do not require solubilization of CREMOPHOR®. Among all these drug carrier systems, liposomes represent a mature technology with a considerable potential for encapsulation of lipophilic molecules like paclitaxel and have been used to formulate a variety of hydrophobic, poorly soluble drugs (Straubinger et al., 1993; Sharma & Straubinger, 1994; Torchilin, 2005a; Torchilin, 2005b). Sharma et al. developed a liposome-based formulation composed of phosphatidylcholine and phosphatidylglycerol for paclitaxel. The in vitro growth-inhibitory activity of liposomal paclitaxel against a variety of tumor cell lines was found to be similar to that of the free drug (Sharma & Straubinger, 1994).

Other active agents including, but not limited to other lipophilic active and/or therapeutic agents, can also be employed with the liposomes disclosed herein. For example, in some embodiments an active agent comprises a chemotherapeutic agent selected from the group consisting of docetaxel, camptothecin, carmustine/BCNU, lomustine/CCNU, vincristine, vinblastine, and doxorubicin. After consideration of the instant disclosure, one of ordinary skill in the art would recognize that one or more of the listed active agents and/or another active agent that is known in the art might be expected to provide a therapeutic benefit, depending on the condition to be treated. As such, the presently disclosed subject matter is not intended to be restricted to only the exemplary active agents listed herein.

II.E. Conjugation Methods

Several approaches are currently available for conjugation of ligands to liposomes. These conjugation methods can be selected based on delivery system requirements like time required for conjugation, stability of conjugate, and/or conjugation efficiency. An exemplary conjugation method occurs rapidly with stable bond formation. The conjugation efficiency can optionally be more than 70%, and in some embodiments this linker does not affect the reactivity of the ligand or stability of the liposomal drug.

A variety of functionalized lipids are available for attaching ligands to liposomes with covalent or non-covalent bonds (see e.g., Pardridge, 1999b; Allen, 2004). Chemical-based linkers employ activating reagents such as maleimidobenzoyl N-hydroxysuccinimide ester (MBS) or 2-iminothiolane (Traut's reagent), which activate primary amino groups on surface lysine (Lys) residues of the ligand. This strategy results in the formation of a stable thioether linkage that comprises a single sulfur atom and generally is not subject to cleavage in vivo (i.e., the resulting thioether bond is stable under physiological conditions). Cleavable bonds like ester and disulfide bond between ligand and liposome have been shown in some instances to be suboptimal for targeted drug delivery to tumors (Martin et al., 1981).

A potential disadvantage with chemical coupling methods can be a low yield in the coupling efficiency. Non covalent coupling methods like avidin-biotin conjugation can be more rapid (Loughrey et al., 1987), more stable, and can be characterized by a higher coupling efficiency (Hansen et al., 1995; Sakahara & Saga, 1999). The type and length of the polymer spacer was also found to potentially influence target recognition and binding in liposomes that already contained PEG-derivatized lipids (Gabizon et al., 1999).

New and relatively simple post-insertion techniques for preparation of targeted liposomes have been reported (Ishida et al., 1999; Allen et al., 2002). According to this method, micellar conjugates of the ligand are co-incubated with pre-formed liposomes to form targeted liposomes. However, this post-insertion technique is usually performed at elevated temperatures (55-60° C.) to accommodate lipid bilayers with higher melting temperatures. Therefore, the denaturation of protein ligands under these conditions can be a concern.

A major drawback of covalent chemical coupling strategies is the difficulty in obtaining reproducible and high coupling efficiencies (Schnyder et al., 2004). In contrast to chemical conjugation methods, the avidin/biotin conjugation method is a noncovalent coupling method. The avidin-biotin interaction is extremely strong with a dissociation constant (K_{d}) in the order of 10⁻¹⁵ M and a dissociation half-life of about 89 days (Sakahara & Saga 1999). The stability of the avidin/biotin bond is very high in blood circulation; however it is labile in tissues (Pardridge, 1999b).

Avidin, an egg white protein, is a 64 kDa homotetramer that has four biotin binding sites per molecule (Green, 1964; Green & Joynson, 1970; Green, 1990). Owing to its cationic nature, avidin is rapidly removed from the plasma compartment following administration in vivo (Green, 1990; Qian, 2002). This rapid plasma clearance results in a reduced plasma area under the concentration curve (AUC) and sub-optimal bioavailability and pharmacokinetics of drug-vector complexes. However, neutral forms of avidin such as streptavidin or neutral light avidin are not as rapidly removed in vivo (Qian, 2002), and do not degrade the pharmacokinetic properties of the conjugate.

Additionally, a compound can be conjugated to a liposome by including within the compound (e.g., at one of the N-terminus and the C-terminus when the compound is a polypeptide) a lipophilic moiety. As used herein, the term “lipophilic moiety” refers to any moiety that targets the compound to a lipid bilayer of a liposome. In some embodiments, the lipophilic moiety causes the compound to be inserted into the lipid bilayer. As would be understood by one of ordinary skill in the art, when the lipophilic moiety inserts into a lipid bilayer, the remainder of the compound can remain outside of the bilayer in a manner similar to how a transmembrane protein is anchored to a cell membrane by virtue of a transmembrane sequence. Thus, the presence of a lipophilic moiety in a compound (e.g., a targeting agent) can result in the compound (e.g., the targeting agent) being anchored to the liposome while at least a region of the compound (e.g., the region of the targeting agent that binds to the binding molecule) remains external to the liposome. As such, in some embodiments a targeting agent can be attached to the liposome by a covalent bind between an atom at the exterior surface of the liposome, and in some embodiments a targeting agent can be attached to the liposome as a consequence of the targeting agent having a lipophilic moiety that anchors the targeting agent to the lipid bilayer of the liposome while exposing the targeting agent to the exterior of the liposome.

### III. Methods for Inhibiting Undesirable Cellular Proliferation

The presently disclosed subject matter also provides methods for inhibiting undesirable proliferation of a glioma cell or of a glioma in a subject. In some embodiments, the methods comprise administering to the subject a composition comprising one or more liposomes comprising an effective amount of an active agent, wherein (i) the active agent has activity in inhibiting undesirable proliferation in the glioma cell or in the glioma; and (ii) the one or more liposomes comprise one or more targeting agents that preferentially or specifically bind to a binding molecule present on the glioma cell or in the glioma, whereby the liposomes target the glioma cell or the glioma to deliver the active agent to the glioma cell or to the glioma.

III.A. Formulations

A composition comprising one or more liposomes comprising an effective amount of an active agent as described herein comprises in some embodiments a composition that includes a pharmaceutically acceptable carrier. Suitable formulations include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The compositions used in the methods can take such forms as suspensions, solutions, or emulsions in oily or aqueous vehicles, and can contain formulatory agents such as suspending, stabilizing, and/or dispersing agents. Alternatively, the liposomes can be in lyophilized form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pre-gelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulfate). The tablets can be coated by methods known in the art. For example, an ACE inhibitor can be formulated in combination with hydrochlorothiazide, and as a pH stabilized core having an enteric or delayed release coating which protects the ACE inhibitor until it reaches the colon.

Liquid preparations for oral administration can take the form of, for example, solutions, syrups, or suspensions, or they can be presented as a dry product for constitution with water or suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions.

III.B. Doses

Actual dosage levels of liposomes in a composition of the presently disclosed subject matter can be varied so as to administer an amount of the active agent(s) that is effective to achieve the desired therapeutic response for a particular subject and/or application. The selected dosage level can depend upon a variety of factors including the activity of the active agent, formulation, the route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of a therapeutically effective dose, as well as evaluation of when and how to make such adjustments, are known to those of ordinary skill in the art.

For administration of a liposome-containing composition as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich et al., 1966). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

For additional guidance regarding formulation and dose, see U.S. Pat. Nos. 5,326,902 and 5,234,933; PCT International Patent Application Publication No. WO 93/25521; Berkow et al., 1997; Goodman et al., 1996; Ebadi, 1998; Katzung, 2001; Remington et al., 1975; Speight et al., 1997; and Duch et al., 1998.

III.C. Routes of Administration

Suitable methods for administering a liposome-containing composition of the presently disclosed subject matter include but are not limited to systemic administration, parenteral administration (including intravascular, intramuscular, intraarterial administration), oral delivery, buccal delivery, subcutaneous administration, inhalation, intratracheal installation, surgical implantation (e.g., intracranial implantation), transdermal delivery, and local injection (e.g., intracranial injection). Where applicable, continuous infusion can enhance drug accumulation at a target site (see e.g., U.S. Pat. No. 6,180,082).

III.D. Combination Therapies

Tumors and/or cancers (e.g., gliomas) can be treated using combination therapies comprising combinations of surgery, radiotherapy, and/or chemotherapy, and/or other therapies include, but not limited to photodynamic therapy (PDT) and immunotherapy (IT). Thus, the presently disclosed subject matter can be employed as a part of a combination therapy. As used herein, the phrase “combination therapy” refers to any treatment wherein the methods and compositions disclosed herein are used in combination with another therapy including, but not limited to radiation therapy (radiotherapy), other chemotherapies, surgical therapy (e.g., resection), PDT, IT, and combinations thereof.

The methods and/or compositions disclosed herein can be employed to enhance the effectiveness of a second treatment such as radiotherapy, another chemotherapy, photodynamic therapy, immunotherapy, or combinations thereof.

III.D.1. Radiation Treatment

In some embodiments, the methods and compositions disclosed herein are employed in a combination therapy with radiation treatment. For such treatment of a tumor (e.g., a glioma), the tumor is irradiated concurrent with, or subsequent to, administration of a composition as disclosed herein. One of skill in the medical art can design, upon consideration of the instant disclosure, an appropriate dosing schedule for treating a subject with radiation in conjunction with the compositions and methods disclosed herein. For example, tumors (e.g., gliomas) can be irradiated with brachytherapy utilizing high dose rate or low dose rate brachytherapy internal emitters.

The composition can be administered beginning, for example, 5, 10, 15, 20, 30, 45, or 60 minutes before the radiation treatment is administered. Additionally, the composition can be administered subsequent to the radiation treatment.

It is understood that since radiotherapy typically is repeated several times in order to affect a maximal response, the administration of the composition can likewise be repeated each time radiotherapy is given. Thus, the time course over which a composition as disclosed herein is administered can comprise in some embodiments a period of several weeks to several months coincident with radiotherapy, but in some embodiments can extend to a period of 1 year to 3 years as needed to effect tumor control. Alternatively, a composition can be administered prior to an initial radiation treatment and then at desired intervals during the course of radiation treatment (e.g., weekly, monthly, or as required).

Subtherapeutic or therapeutic doses of radiation can be used for treatment of a tumor and/or a cancer (e.g., a glioma) as disclosed herein. In some embodiments, a subtherapeutic or minimally therapeutic dose (when administered alone) of ionizing radiation is used. For example, the dose of radiation can comprise in some embodiments at least about 2 Gy ionizing radiation, in some embodiments about 2 Gy to about 6 Gy ionizing radiation, and in some embodiments about 2 Gy to about 3 Gy ionizing radiation. When radiosurgery is used, representative doses of radiation include about 10 Gy to about 20 Gy administered as a single dose during radiosurgery or about 7 Gy administered daily for 3 days (about 21 Gy total). When high dose rate brachytherapy is used, a representative radiation dose comprises about 7 Gy daily for 3 days (about 21 Gy total). For low dose rate brachytherapy, radiation doses typically comprise about 12 Gy administered twice over the course of 1 month. ¹²⁵I seeds can be implanted into a tumor can be used to deliver very high doses of about 110 Gy to about 140 Gy in a single administration.

Radiation can be localized to a tumor using conformal irradiation, brachytherapy, stereotactic irradiation, intensity modulated radiation therapy (IMRT), and/or can be localized to a tumor by employing vectors that comprise, but are not limited to, proteins, antibodies, liposomes, lipids, nanoparticles, and combinations thereof that target the tumor. The threshold dose for treatment can thereby be exceeded in the target tissue but avoided in surrounding normal tissues.

Radiation can also comprise administration of internal emitters, for example ¹³¹I, ³²P, ¹²⁵I, iridium, and cesium. Internal emitters can be encapsulated for administration or can be loaded into a brachytherapy device.

Radiotherapy methods suitable for use in the practice of presently disclosed subject matter can be found in Leibel & Phillips, 1998, among other sources.

III.D.2. Other Chemotherapy Treatments

In some embodiments, the methods and compositions disclosed herein are employed in a combination therapy with additional chemotherapy treatments. Particular chemotherapeutic agents are generally chosen based upon the type of tumor to be treated, and such selection is within the skill of the medical professional.

Chemotherapeutic agents are generally grouped into several categories including, but not limited to DNA-interactive agents, anti-metabolites, tubulin-interactive agents, hormonal agents, and others such as asparaginase or hydroxyurea. Each of the groups of chemotherapeutic agents can be further divided by type of activity or compound. For a detailed discussion of various chemotherapeutic agents and their methods for administration, see Dorr et al., 1994, herein incorporated by reference in its entirety.

In order to reduce the mass of the tumor and/or stop the growth of the cancer cells, a chemotherapeutic agent should prevent the cells from replicating and/or should interfere with the cell's ability to maintain itself. Exemplary agents that accomplish this are primarily the DNA-interactive agents such as Cisplatin, and tubulin interactive agents.

DNA-interactive agents include, for example, alkylating agents (e.g., Cisplatin, Cyclophosphamide, Altretamine); DNA strand-breakage agents (e.g., Bleomycin); intercalating topoisomerase II inhibitors (e.g., Dactinomycin and Doxorubicin); non-intercalating topoisomerase II inhibitors (e.g., Etoposide and Teniposide); and the DNA minor groove binder Plicamycin.

Generally, alkylating agents form covalent chemical adducts with cellular DNA, RNA, and/or protein molecules, and with smaller amino acids, glutathione, and/or similar biomolecules. These alkylating agents typically react with a nucleophilic atom in a cellular constituent, such as an amino, carboxyl, phosphate, or sulfhydryl group in nucleic acids, proteins, amino acids, or glutathione.

Anti-metabolites interfere with the production of nucleic acids by either of two major mechanisms. Some of the drugs inhibit production of deoxyribonucleoside triphosphates that are the immediate precursors for DNA synthesis, thus inhibiting DNA replication. Some of the compounds are sufficiently like purines or pyrimidines to be able to substitute for them in the anabolic nucleotide pathways. These analogs can then be substituted into the DNA and RNA instead of their normal counterparts.

Hydroxyurea appears to act primarily through inhibition of the enzyme ribonucleotide reductase.

Asparaginase is an enzyme which converts asparagine to nonfunctional aspartic acid and thus blocks protein synthesis in the tumor.

Paclitaxel is a tubulin interactive agent. Tubulin interactive agents act by binding to specific sites on tubulin, a protein that polymerizes to form cellular microtubules. Microtubules are critical cell structure units. When the interactive agents bind on the protein, the cell can not form microtubules. Other tubulin interactive agents include Vincristine and Vinblastine, which are both alkaloids.

Adrenal corticosteroids are derived from natural adrenal cortisol or hydrocortisone. They are used because of their anti-inflammatory benefits as well as the ability of some to inhibit mitotic divisions and to halt DNA synthesis. These compounds include Prednisone, Dexamethasone, Methylprednisolone, and Prednisolone.

The hormonal agents and leutinizing hormones are not usually used to substantially reduce the tumor mass. However, they can be used in conjunction with the chemotherapeutic agents. Hormonal blocking agents are also useful in the treatment of cancers and tumors. They are used in hormonally susceptible tumors and are usually derived from natural sources. These include, but are not limited to estrogens and conjugated estrogens, progestins, and androgens. Leutinizing hormone releasing hormone agents or gonadotropin-releasing hormone antagonists are used primarily the treatment of prostate cancer. These include leuprolide acetate and goserelin acetate. They prevent the biosynthesis of steroids in the testes. Other anti-hormonal agents include anti-estrogenic agents, anti-androgen agents, and anti-adrenal agents such as mitotane and aminoglutethimide(3-(4-aminophenyl)-3-ethyl-piperidine-2,6-dione).

### EXAMPLES

The following Examples have been included to illustrate modes of the presently disclosed subject matter. Certain aspects of the following Examples are described in terms of techniques and procedures found or contemplated by the present co-inventors to work well in the practice of the presently disclosed subject matter. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the presently disclosed subject matter.

### Introduction to the Examples

Liposomes are versatile carriers for targeted drug delivery by the intravenous route. Various types of liposomal formulations have been extensively used as carriers for increasing the therapeutic index of cytotoxic drugs (Lasic et al., 1991; Lasic, 1994; Allen, 1997; Lasic, 1997; Torchilin, 2004; Torchilin, 2005a; Torchilin, 2005b). A concern associated with the use of liposomes for targeting tumor cells in extravascular sites is the rapid clearance from the systemic circulation. These liposomal carriers are subjected to rapid clearance by circulating phagocytes and by macrophages of liver and spleen (Harasym et al., 1998). Short circulation life time decreases passive accumulation of targeted liposomes in the tumor tissue. Long circulation lifetimes play a role in target site accumulation of liposomal drugs, which is desirable for target cell access and increased circulation half-life can result in increased accumulation within a target site.

One of the strategies to circumvent this problem is to design long circulating liposomes with steric stabilization. Surface modification of liposomes with hydrophilic polymers such as polyethylene glycol (PEG) resulted in decreased recognition and subsequent phagocytosis by cells of the mononuclear phagocytic system (MPS). The concentration of PEG lipid in the liposomes and size of liposomes are two parameters that can be optimized for increasing the circulation times of liposomes for achieving anticipated increase in localization within the target tumor site. An understanding of the liposome targeting to tumor, its blood circulation, and distribution to various organs can be achieved by labeling with fluorescent dyes.

Disclosed herein is the preparation and characterization of fluorescently labeled conventional, sterically stabilized, and targeted liposomes. The blood circulation half-life in rats for fluorescent-labeled formulations was determined. The sterically stabilized liposomes prepared using 5% PEG and about 100 nm in size showed a pronounced increase in the blood residence time (17 hours) with a significant decrease in uptake by RES when compared with conventional liposomes. Also disclosed is the localization of the fluorescent liposomes in a flank C6 glioma xenograft bearing nude mice using non-invasive near infrared fluorescence imaging. Also provided is evidence of selective tumor localization of targeted liposomal formulations in comparison to non targeted liposomes and solution formulation as controls.

### Materials and Methods for Examples 1-4

Materials. Hydrogenated soybean phosphatidylcholine (HSPC), 1,2-distearoyl-glycero-3-phosphoethanolamine-N-[PEG(2000)] conjugate (DSPE-PEG), and DSPE-PEG-biotin were from Northern Lipids Inc., Vancouver, Canada. Cholesterol and biotinylated transferrin were obtained from Sigma (St. Louis, Mo., United States of America). DIR and Dil were purchased from Invitrogen Corp. (Carlsbad, Calif., United States of America). These chemicals were used as received. All other chemicals and solvents were of analytical grade.

Liposome Preparation: Liposomes were produced by lipid hydration method followed by extrusion method using hydrogenated soy phosphatidylcholine (HSPC), cholesterol (95:5 molar ratios). The Dil or DIR dyes were entrapped in the lipid bilayers due to its lipophilic nature. For preparation of long circulating liposomes (LCL), polyethylene glycol-2000 grafted distearoyl phosphatidyl ethanolamine (DSPE-PEG2000) was incorporated. For preparation of transferrin conjugated liposomes (Tf-LCL), a portion of DSPE-PEG2000 (0.01 mol %) was replaced with DSPE-PEG2000-biotin. Transferrin (Tf) was noncovalently conjugated at the distal end of DSPE-PEG2000-biotin via a streptavidin-biotin bond. The dye loading was quantified via spectrofluorometry. A centrifugal ultrafiltration device (Centricon 100, MWCO 100 kDa; Millipore, Bedford, Mass., United States of America) was used to separate the free dye from the dye entrapped in the liposomes. Free and total dye concentration in the liposomes was determined using a FLx800 fluorometric plate reader (BioTek Instruments, Inc, Winooski, Vt., United States of America) after 70% isopropanol extraction. The percent dye entrapped in the liposomes was calculated from the free and total dye in the liposomes. Liposome Size, Morphology, and Zeta Potential: The size analyses of liposomes were performed by the dynamic light scattering technique using a Malvern zeta-sizer nano particle size analyzer (Malvern Instruments, Malvern, United Kingdom). 25 μL of this sample were diluted to 1 mL with water for injection for particle size determination. The diluted aqueous sample (1 mL) was added to a 2 mL cuvette and the particle size analysis was performed in triplicate. The average particle size was calculated form the results. The transmission electron microscopic (TEM) studies were carried out using 3 mm Forman coated copper grid (400 mesh) at 60 KV using negative staining by 2% uranyl acetate at 200,000× magnifications on a JEOL 1200EX TEM (JEOL Ltd., Tokyo, Japan).

Quantification of transferrin conjugation: A sensitive gel filtration chromatographic method was developed and validated to quantitate transferrin. The gel filtration chromatographic system consisted of a Waters 600 controller, Waters 717 plus auto sampler and a Waters 2996 photodiode array detector. Data were acquired and processed with Waters Millennium 32 software (version 4.0). Gel filtration chromatographic separation was achieved on a TSK Gel G3000 SWXL (30 cm×7.8 mm, 5 micron) column from Tosoh bioscience (South San Francisco, Calif., United States of America). The isocratic mobile phase consisting of 0.5 N phosphate buffered saline (pH 7.2) was pumped at a flow rate of 0.5 mL/min with an injection volume of 30 μL. Transferrin (retention time, 4.8 min) was monitored at 220 nm with a photodiode array detector. All analyses were performed in triplicate, and the mean peak area was used to determine the concentration of transferrin in the samples. Free Tf was separated from the liposome-entrapped fraction using a Centricon centrifugal filter device (Centricon 100, MWCO 100 kDa; Millipore, Bedford, Mass., United States of America). An aliquot of the liposome dispersion (100 μL) was diluted to 1 mL with hydration buffer (phosphate buffered saline pH 7.2), and this sample was transferred to the centrifugal filter device. The sample was centrifuged at 5000 rpm for 30 minutes in a fixed-angle centrifuge. Free transferrin in the filtrate was then determined using high performance gel filtration chromatography (HPGFC). Subtraction of free transferrin from the total amount added gave the amount of liposome-conjugated Tf. Tf estimations were done in triplicate, and the values were reported as mean±SD.

Determination of blood circulation time of formulations: Fluorescent liposome formulations or fluorochrome in solution formulations were administered systemically to Sprague Dawley rats via tail vein. At predetermined time points, blood samples were withdrawn. The serum was separated and treated with ice-cold 70% isopropyl alcohol followed by centrifugation at 12,000×g for 5 min for extraction of dye. Fluorescence intensity in the clear supernatant was determined using a FLX800 fluorometric plate reader (BioTek Instruments, Inc, Winooski, Vt., United States of America; Excitation 555/Emission: 575 nm).

Cranial window preparation for intravital fluorescence imaging (IVM) of blood circulation time of liposomes in rats: Cranial window preparation was made according to published method (Gaber et al., 2004). Briefly, a glass cranial window (0.8×0.8 mm), extending from the bregma to lambda sutures and centered on the sagittal suture, was placed and fixed over the surgically exposed cerebral cortex. Before surgery, the animals were anesthetized with an i.m. injection (1 mL/Kg) of ketamine/Xylazine solution (87 mg ketamine/mL+13 mg Xylazine/mL). Animals were placed in a stereotaxic frame (Kopf Instruments, Tujunga, Calif., United States of America), and their body temperature was maintained at approximately 37° C. using a heating pad. One dose of the chloromycetin (50 mg/kg body weight) was given before surgery. The scalp and underlying soft tissue over the parietal cortex were removed. A rectangular cranial window was made using a low-speed dental drill (0-8000 rpm, DENTSPLY Professional, Des Plaines, Ill., United States of America) along with artificial cerebral-spinal fluid irrigation. The dura mater was cleared using iris microscissors. A glass plate was fixed to the bone using cyanoacrylate glue. After one week of recovery from the surgery, animals were ready for data collection. Fluorescent liposome formulation containing 40 μg of Dil was administered systemically to rats via tail vein and subjected to fluorescence excitation. The emitted light through the cranial window was detected by a COOLSNAP® monochrome camera (Princeton Instruments Inc., Trenton, N.J., United States of America) attached to a fluorescence intravital microscope at 0, 1, 4, 24 and 48 hour time points. Images acquired were processed with METAMORPH® software (version 6.2; Molecular Devices, Sunnyvale, Calif., United States of America). The fluorescence produced from the liposomes was observed through this glass window using an intravital microscope (model MM-11, Nikon Inc.; Melville, N.Y., United States of America) with a DII filter (Excitation 555/Emission: 575 nm).

### Example 1

### Preparation and Characterization of Fluorescent Liposomes

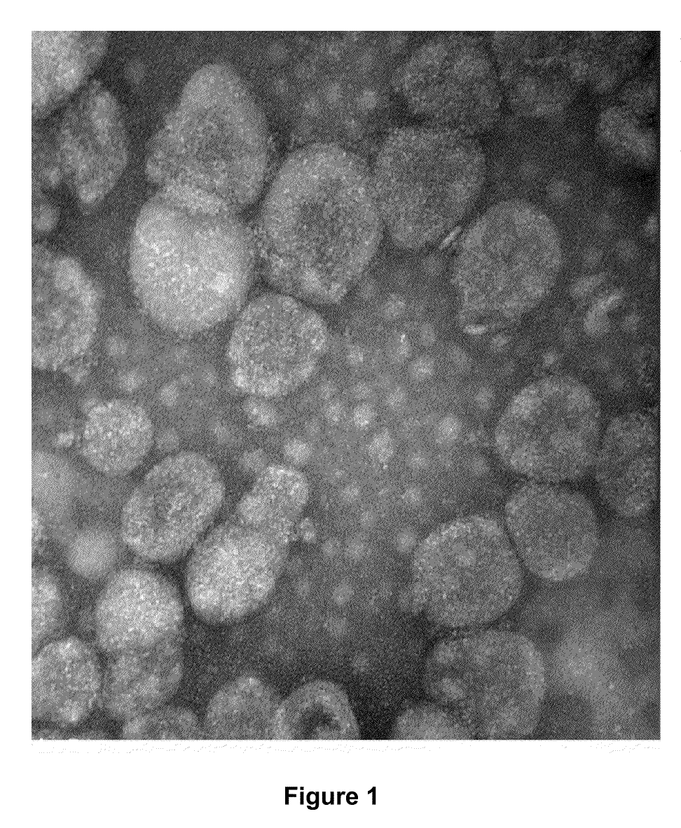

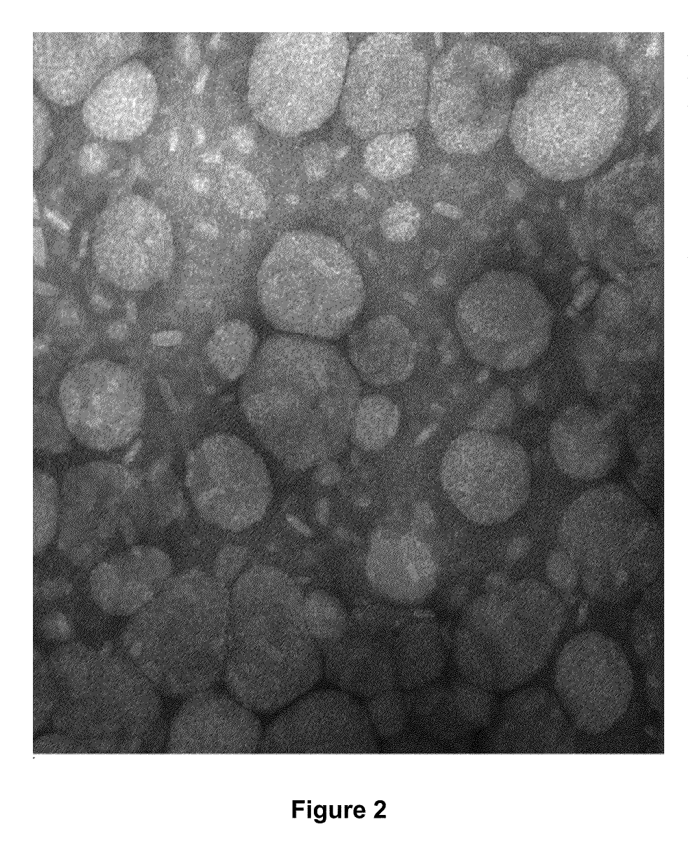

Fluorescent-labeled liposomal formulations were prepared by Bangham method followed by polycarbonate membrane extrusion. To attach the transferrin ligand to the liposomes, the streptavidin-biotin conjugation method was used (Loughrey et al., 1987). Dye incorporation efficiency was determined by subtracting the free dye fraction from the total dye and was found to be 95.0±3%. The non-conjugated liposome formulation showed average vesicle sizes of 81±7 nm with a unimodal distribution. The covalent coupling of Tf to the liposome surface led to a slight increase in diameter to about 115±11 nm. This slight increase in size was most probably due to the attachment of Tf to the liposome surface, which somewhat increased the hydrodynamic diameter of liposomes. The TEM images (see FIGS. 1 and 2) revealed that the long circulating liposomes are round and spherical in shape. The zeta potential of Tf conjugated and non-conjugated long circulating liposomes was −18.0±1.2 mV. The Tf conjugation of LCL resulted in slight increase in the zeta potential of liposomes to −25.5±1.5 mV.

The transferrin conjugation was studied using TEM. The surface of non-conjugated liposomes was relatively smooth (see FIG. 2). However, the surface of Tf conjugated liposomes was more granular (see FIG. 1). The total amount of the liposome attached transferrin in the formulations was determined by using the high performance gel filtration chromatography (HPGFC). Based on free Tf concentration from a typical formulation, about 72% of Tf was coupled to the liposomes. It was found by this method that about 4 μg of the Tf was bound to 1 μM of the total lipid; this corresponds to approximately 5 Tf molecules per 100 nm liposome.

### Example 2

### Determination of Blood Circulation Time

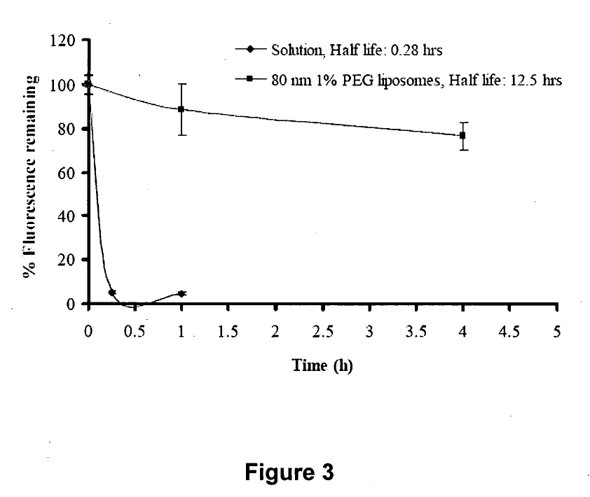

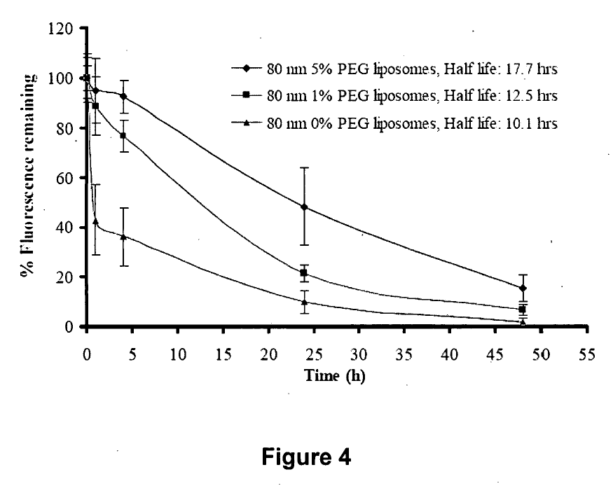

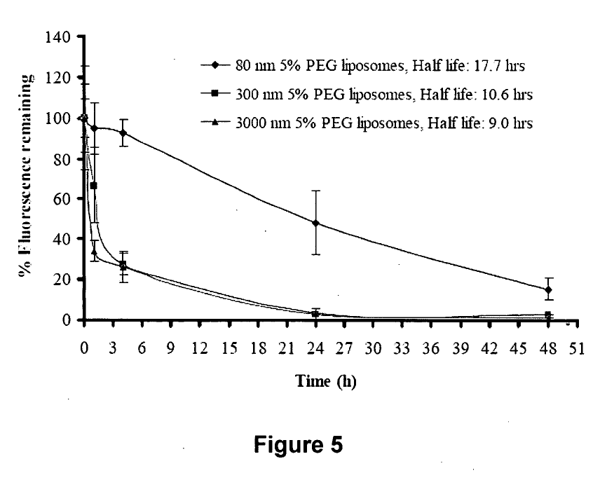

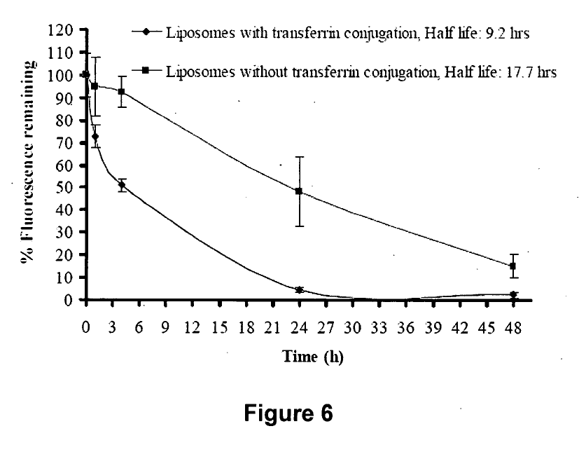

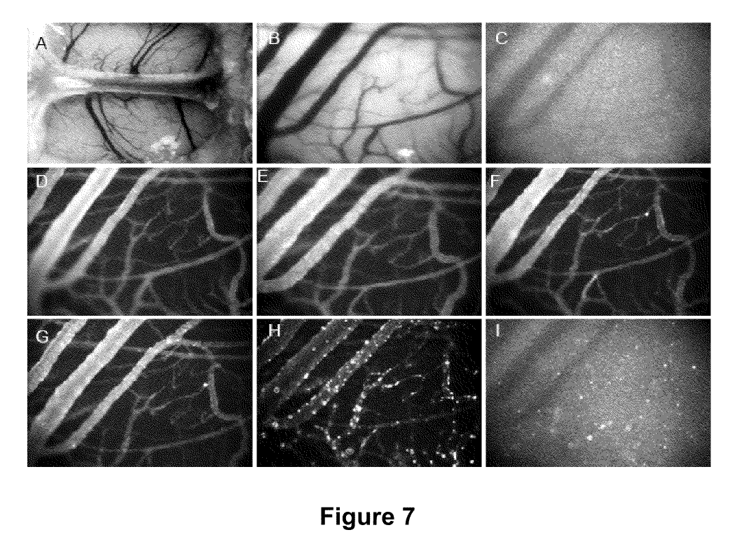

The blood circulation time of fluorescent-labeled formulations was determined using blood sampling method. In this experiment, fluorescent-labeled formulations were injected into Sprague Dawley rats via tail vein injection. The fluorescence in clear serum samples was measured with a spectrofluorimetric plate reader. As shown in the FIG. 3, blood circulation half-life of long circulating liposomes (with 1% DSPE-PEG2000) in the brain microvasculature was about 12 hours, whereas the Dil solution formulation (Dil in water containing 3% CREMOPHOR® EL) was cleared within 1 hour after administration. An increase in DSPE-PEG2000 concentration in the liposomes prolonged the in vivo circulation, and maximum in vivo circulation half-life of about 17 hours was achieved with a formulation containing 5% of DSPE-PEG2000 in total lipids (see FIG. 4). Liposomes larger than 300 nm showed a significant reduction in blood circulation time when compared to 100 nm liposomes (see FIG. 5). The conjugation of Tf on the distal end of PEG chains led to a reduction in blood circulation half-life (˜9 hours) as compared to LCL (˜17 hours; see FIG. 6). Blood circulation time of transferrin conjugated liposomes was also determined by real-time monitoring of circulating fluorescent labeled liposomes in rat brain pial vessels using intravital microscopy. The blood circulation half-life of the transferrin-conjugated liposomes determined with the intravital microscopic technique was comparable to that determined with the blood sampling method. Intravital microscopic imaging of transferrin-conjugated liposomes also revealed their probable interaction with the brain microvasculature after 24 hour time point (see FIGS. 7A-7I). However, these liposomes appeared to clear from the vasculature into the surrounding brain tissue by the 48 hour time point.

### Example 3

### NIRF Imaging of Liposomal Tumor Localization

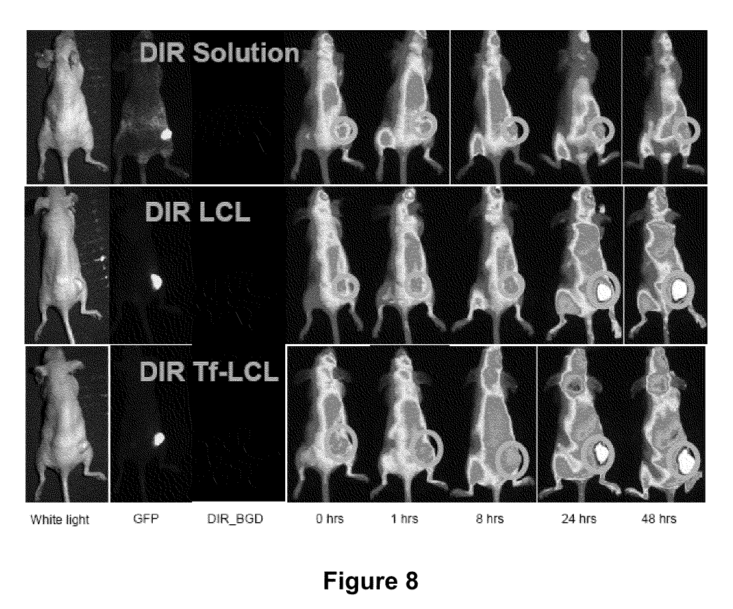

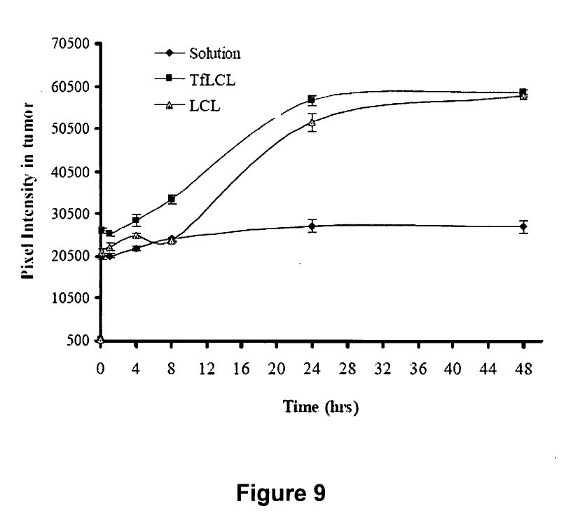

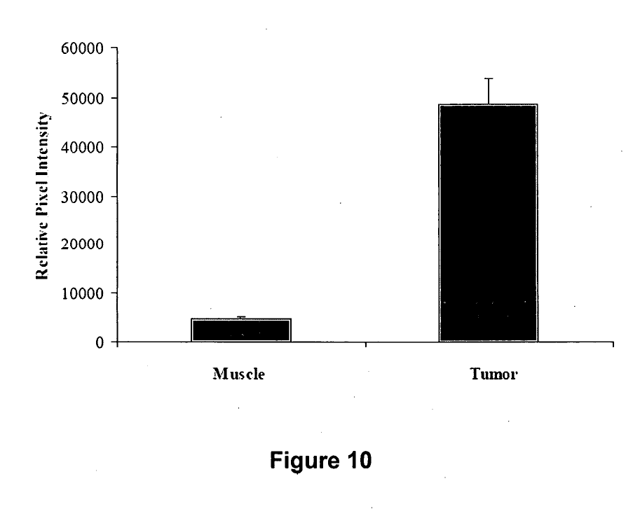

An optical whole-body imaging technique was employed for monitoring tumor localization of liposomes in mice. DIR has absorption and fluorescence maxima at 750 and 782 nm, respectively, which can prevent issues related to autofluorescence in living tissues. This facilitates to get a significant signal with very low background noise level. A charge-coupled device (CCD) based camera is used for non-invasive whole-body imaging of DI R-labeled liposome localization in live animals with tumors. This technique can potentially visualize many types of delivery systems or cells labeled with a near infrared (NIR) fluorescent tag. Tumor accumulation and the tumor-to-muscle accumulation ratio of liposomal formulation were evaluated using a non-invasive NIRF optical imaging method. The data showing that the LCL and Tf-LCL accumulate in C6 glioma flank tumors at higher concentrations when compared to solution formulation is presented in FIG. 8. Tf-LCL accumulates in C6 glioma tumors more efficiently when compared to muscle tissue (see FIG. 9). The results obtained showed that the long circulating liposomal formulations stay in the tumor for a longer time (˜48 hours post-injection). The tumor targeting index (see FIG. 10) was found to be 10.59±1.08.

### Example 4

### C6 GFP Flank Glioma Tumor Model and NIRF Imaging of Liposomal Tumor Localization

The effect of liposome size on tumor targeting potential was determined in a similar C6 glioma tumor model. C6-GFP cells in exponential growth were harvested with 0.25% trypsin with EDTA for 5 min at 37° C. The cells were centrifuged for 5 min at 1,000 RPM. The pellets were resuspended in sterile phosphate buffered saline (PBS), at a concentration of 100,000,000/mL and placed on ice. Adult female CD1 nu/nu mice (weight: 25-30 g each) were used for all studies and handled in accordance with protocols approved by the Animal Care and Use Committee at the University of Tennessee Health Science Center. Mice were anesthetized with intraperitoneal injection of ketamine/xylazine at a dosage of 8.7/1.3 mg/100 g body weight. To create flank glioma tumors, 4,000,000 C6-GFP glioma cells in 200 μL of phosphate buffered saline were injected subcutaneously into the flank using a 27½ G needle. Tumor growth was measured on every 3rd day with a vernier caliper, and tumor volume was measured using the formula

W²×L/0.52.

W refers to width and L is the length of the tumor. Near infrared whole body optical images of mice were taken with a COOLSNAP® CCD camera (Princeton Instruments Inc., Trenton, N.J., United States of America) using GFP filter (excitation: 475 nm and emission: 510 nm). Acquired images were processed for measuring the pixel intensity of the GFP fluorescence from the tumors using the METAMORPH® software (version 6.2; Molecular Devices, Sunnyvale, Calif., United States of America) for determining the C6 GFP tumor area.

After 18 days of tumor cell inoculation, animals were injected retroorbitally with DIR labeled (5 μg of DIR in 50 μL) formulations (n=3 per group). The area and pixel intensity of the dye in the tumor was compared with the background intensity in the surrounding normal tissue using non-invasive optical imaging with a COOLSNAP® CCD camera (Princeton Instruments Inc., Trenton, N.J., United States of America) with a DIR filter (excitation: 750 nm and emission: 782 nm; Omega optical, Brattleboro, Vt., United States of America) at 0, 1, 4, 6, 8, 24 and 48 hours after injection. Acquired images were processed for measuring the pixel intensity of the DIR fluorescence from the tumors using the METAMORPH® software for determining the C6 GFP tumor area. Tumor to muscle accumulation ratio of DIR dye labeled formulations was determined to calculate the tumor targeting index. After 48 hours post-injection, animals were anesthetized and subjected to transcardiac perfusion, first with 20 ml of normal saline, and then with the same amount of 4% paraformaldehyde to fix the tissues. Optical images of isolated organs (tumor, brain, liver, and spleen) were taken with a CCD camera using white light, GFP (excitation: 475 nm and emission: 510 nm) and DIR (excitation: 750 nm and emission: 782 nm) filters for visualization of tumor area and DIR dye localization in tissue. Acquired images were then processed for measuring the pixel intensity of the GFP and DIR fluorescence from the tumors using the METAMORPH® software for determining the C6 GFP tumor area and DIR localization respectively.

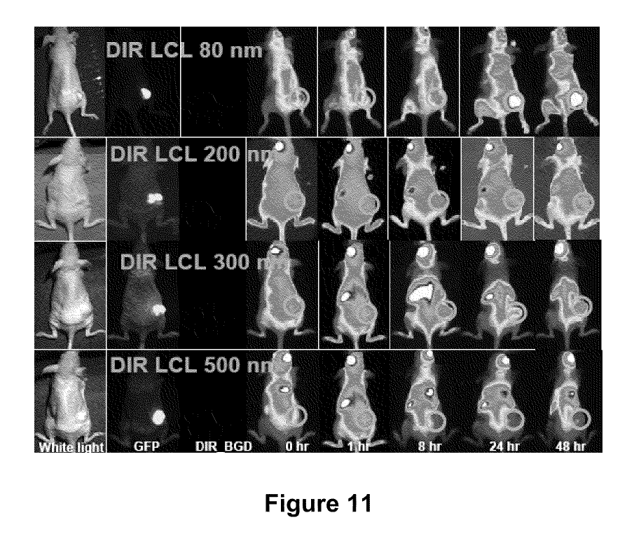

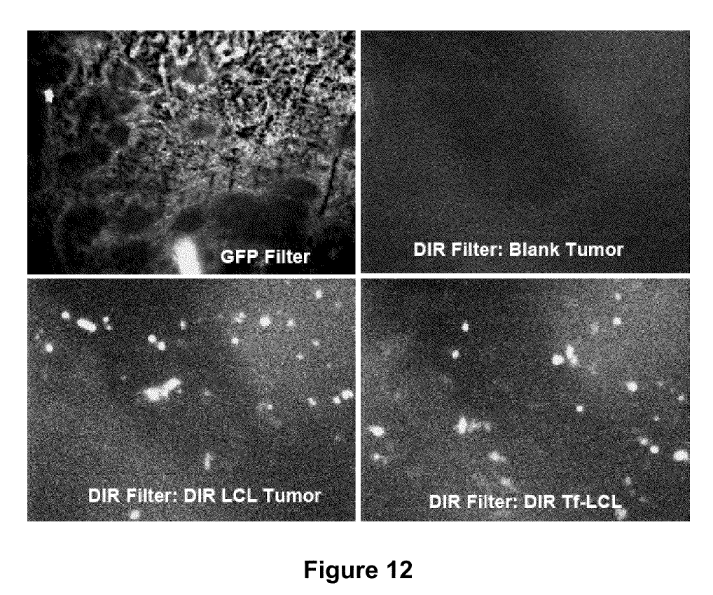

The results show that about 100 nm liposomes can localize in the tumors with high selectivity. On the other hand, liposomes larger than 300 nm showed very limited to no localization of liposomes in the tumor as shown in the FIG. 11. However, higher accumulation was observed in the liver and spleen (see FIG. 11). This might be due to rapid clearance of larger liposomes by these organs. C6 GFP flank tumor sections from the tumor targeting study revealed the presence of DIR fluorescence with 100 nm long circulating liposomes and transferrin conjugated long circulating liposomes (see FIG. 12). The sterically stabilized liposomes prepared using 5% PEG (about 100 nm size) showed a pronounced increase in the blood residence time (17 hours) with a significant decrease in uptake by the RES when compared with conventional liposomes. Selective tumor localization of transferrin targeted liposomal formulations was also achieved when compared to non-targeted liposomes and solution formulation as controls.

These findings indicate that preferential targeting to glioma tumors can be achieved with about 100 nm long circulating liposomes with transferrin conjugation. Transferrin conjugated liposomal delivery system can be a potential drug carrier for glioma tumors targeting.

### Materials and Methods for Examples 5-7

Materials: Egg phosphatidylcholine (EPC), Hydrogenated soybean phosphatidylcholine (HSPC), 1,2-distearoyl-sn-glycero-3-phosphoethanolamine-N-[PEG(2000)] conjugate (DSPE-PEG), and DSPE-PEG-biotin were from Northern Lipids Inc., Vancouver, Canada. Cholesterol and biotinylated Transferrin were obtained from Sigma (St. Louis, Mo., United States of America). These chemicals were used as received. Paclitaxel was purchased from 21CEC Pharma, East Sussex, United Kingdom. All other chemicals and solvents used were of analytical grade.

Preparation and characterization of liposomes: Liposomes were produced by the lipid hydration method followed by extrusion. The liposomes contained egg phosphatidylcholine (EPC), hydrogenated soy phosphatidylcholine (HSPC), and cholesterol (75:15:5 molar ratios). Paclitaxel was entrapped in the lipid bilayers due to its lipophilic nature. For preparation of long circulating liposomes (LCL), poly ethylene glycol-2000-grafted distearoyl phosphatidyl ethanolamine (DSPE-PEG2000) was incorporated. For preparation of Tf-LCL a part of DSPE-PEG2000 (0.01 mol %) was replaced with DSPE-PEG2000-biotin and then transferrin (Tf) was non-covalently conjugated at the distal end of DSPE-PEG2000-biotin via streptavidin-biotin bond.

Quantification of paclitaxel: A sensitive reverse phase HPLC method was developed and validated to quantitate paclitaxel in liposome formulations. Chromatographic system consisted of a Waters 600 controller, Waters 717 plus auto sampler and a Waters 2996 photodiode array detector. Data were acquired and processed with Waters Millennium 32 software (version 4.0). Chromatographic separation was achieved on a NOVAPAK® C18 reverse phase column (3.9×150 mm) from Waters Corp. (Milford, Mass., United States of America). The isocratic mobile phase consisting of acetonitrile and water (55:45, v/v) was pumped at a flow rate of 0.7 mL/min with an injection volume of 20 μL. Paclitaxel (retention time, 3.8 min) was monitored at 230 nm with a photodiode array detector. Prior to HPLC analysis, the formulation samples were treated with methanol for paclitaxel extraction. All analyses were performed in triplicate, and the mean peak area was used to determine the concentration of paclitaxel in the samples. Drug loading was quantified with high-performance liquid chromatography (HPLC). A centrifugal ultrafiltration device (Centricon 100, MWCO 100 kDa; Millipore, Bedford, Mass., United States of America) was used to separate free paclitaxel from the paclitaxel entrapped in the liposomes in the finished product. Total paclitaxel concentration in the liposomes was determined using HPLC after methanol extraction. The percent paclitaxel entrapped in the liposomes was calculated from the free and total paclitaxel in the liposomes.

Liposome size, morphology and zeta-potential: The size analysis of liposomes was performed by the dynamic light scattering technique using a Malvern zeta sizer nano particle size analyzer (Malvern Instruments, Malvern, United Kingdom). A 25 μL of this sample was diluted to 1 mL with water for injection for particle size determination. The diluted aqueous sample (1 mL) was added to a 2 mL cuvette and the analysis was performed in triplicate and the average particle size was calculated from the result. The transmission electron microscopic (TEM) studies were carried out using 3 mm Forman coated copper grid (400 mesh) at 60 KV using negative staining by 2% uranyl acetate at 200,000× magnifications on a JEOL 1200EX TEM (JEOL Ltd., Tokyo, Japan).

Quantification of transferrin conjugation: A sensitive gel filtration chromatographic method was developed and validated to quantitate transferrin. The gel filtration chromatographic system consisted of a Waters 600 controller, Waters 717 plus auto sampler and a Waters 2996 photodiode array detector. Data were acquired and processed with Waters Millennium 32 software (version 4.0). Gel filtration chromatographic separation was achieved on a TSK Gel G3000 SWXL (30 cm×7.8 mm, 5 micron) column from Tosoh Bioscience LLC (Montgomeryville, Pa., United States of America). The isocratic mobile phase included 0.5 N phosphate buffered saline (pH 7.2) pumped at a flow rate of 0.5 mL/min with an injection volume of 30 μL. Transferrin (retention time, 4.8 min) was monitored at 220 nm with a photo diode array detector. All analyses were performed in triplicate, and the mean peak area was used to determine the concentration of transferrin in the samples.

Free Tf was separated from the liposome-entrapped fraction using a Centricon centrifugal filter device (Centricon 100, MWCO 100 kDa; Millipore, Bedford, Mass., United States of America). An aliquot of the liposome dispersion (100 μL) was diluted to 1 mL with hydration buffer (phosphate buffered saline pH 7.2). This sample was transferred to the centrifugal filter device. The sample was centrifuged at 5000 rpm for 30 minutes in a fixed-angle centrifuge. Free transferrin in the filtrate was then determined using high performance gel filtration chromatography (HPGFC). Subtraction of free Tf from the amount added gave the amount of liposome-conjugated Tf. Tf estimations were done in triplicate, and the values were reported as mean±SD.

C6 GFP Flank Glioma Tumor Xenograft Model and Non-invasive Imaging of Tumor Growth using NIRF: C6-GFP glioma cells in their exponential growth phase were harvested with EDTA/Trypsin for 5 min at 37° C. The cells were centrifuged for 5 min at 1,000 RPM. The pellets were resuspended in sterile phosphate buffered saline (PBS), at a concentration of 100,000,000/mL and placed on ice. Adult CD1 nu/nu mice (weight 25-30 g each) were used for all studies and handled in accordance with protocols approved by the Animal Care and Use Committee at the University of Tennessee Health Science Center. Mice were anesthetized with an intraperitoneal injection of ketamine/xylazine at a dosage of 8.7/1.3 mg/100 g body weight. To create the flank glioma tumor model, 4,000,000 C6-GFP glioma cells in 200 μL of phosphate buffered saline were injected into the flank using a 27½ G needle. Tumor growth was measured on every 3rd day with a vernier caliper, and tumor volume was measured using the formula

W²×L/0.52.

W refers to width and L is the length of the tumor. Near infrared whole body optical images of mice were taken with a CCD camera (Princeton Instruments Inc. Trenton, N.J., United States of America) using a GFP filter (excitation: 475 nm and emission: 510 nm). Acquired images were processed for measuring the pixel intensity of the GFP fluorescence from the tumors using the METAMORPH® software (version 6.2) for determining the C6 GFP tumor area.

Antitumor efficacy of paclitaxel in liposomes—NIRF imaging analysis: Five days after tumor cell inoculation, animals were assigned randomly in to 4 groups (n=4-5 per group). The groups included no treatment control, paclitaxel in transferrin conjugated long circulating liposomes (Tf-LCL), paclitaxel in long circulating liposomes (LCL) and paclitaxel in water containing 8.3% of CREMOPHOR® EL and 8.2% ethanol. After 5 days of unrestricted tumor growth, animals were treated with 2 mg/Kg of paclitaxel on every third day (about 13 days) till the end of experiment. Injections were made using the retro orbital route of administration. For monitoring changes in tumor volume, tumor growth was measured every 3rd day with a vernier caliper, and tumor volume was measured as described in previous section. Near infrared whole body optical imaging was also explored for monitoring the biodistribution and therapeutic effect. Optical images of mice were taken with a CCD camera (Princeton Instruments Inc., Trenton, N.J., United States of America) using GFP filter to visualize the tumors. Acquired images were processed using the METAMORPH® software (version 6.2) for determining the C6 GFP tumor area. On the day 18th of tumor implantation, animals were anesthetized and subjected to transcardiac perfusion first with 20 ml of normal saline and then with same amount of 4% paraformaldehyde to fix the tissue. Acquired images were processed for measuring the pixel intensity of the GFP fluorescence from the tumors using the METAMORPH® software (version 6.2) for determining the C6 GFP tumor area.

Four different mouse treatment groups were observed: one group (n=10) did not receive any treatment and served as control, another group (n=4) was injected with CREMOPHOR® EL micellar solubilized paclitaxel at a concentration of 2 mg/Kg body weight, another group (n=4) received 2 mg/Kg of paclitaxel in long circulating liposomes (LCL) and the last group (n=4) received 2 mg/Kg of paclitaxel in Tf conjugated long circulating liposomes. The tumor growth delay was defined as the time required for a treated tumor to reach a specific volume (130 mm³) minus the time for the untreated tumor to reach that same volume.

Statistics: The in vivo tumor localization and antitumor efficacy data were compared using one-way analysis of variance to determine significant differences among experimental groups. All values of P≦0.05 were considered statistically significant.

### Example 5

### Preparation and Characterization of Transferrin-Conjugated Liposomal Paclitaxel

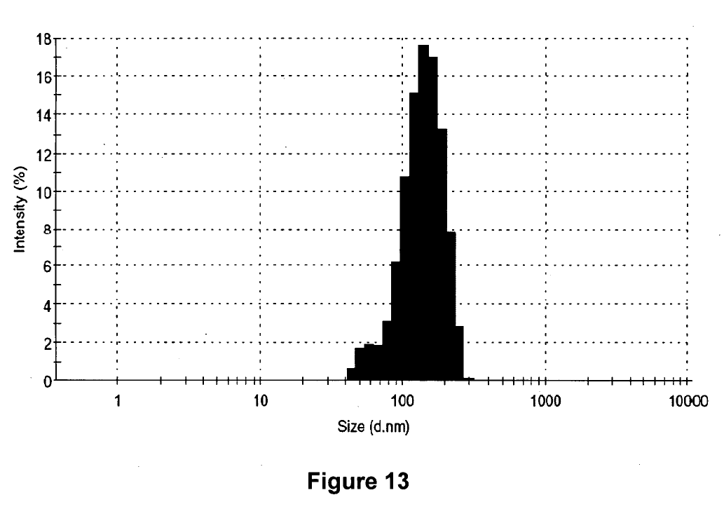

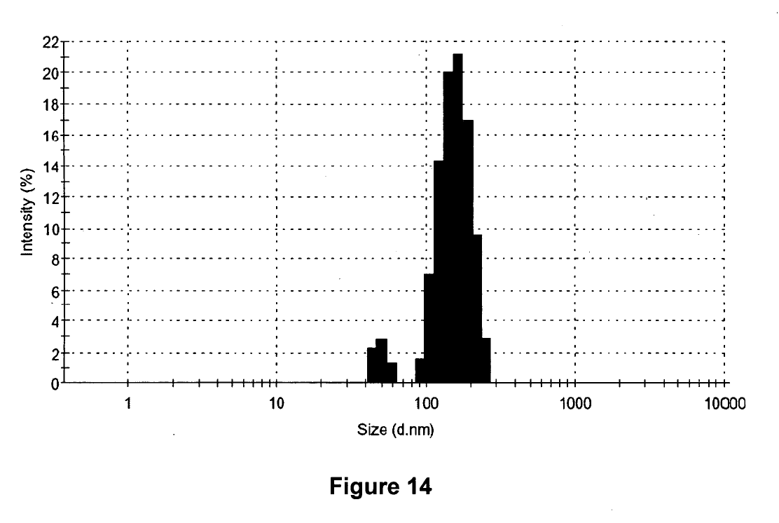

Paclitaxel incorporation efficiency was determined by subtracting the free drug fraction from the total was found to be 98.0±2%. The non-targeted liposome formulation showed average vesicle sizes of 133±15 nm with unimodal distribution. The covalent coupling of Tf to the liposome surface led to a slight increase in diameter to about 141±20 nm (see FIGS. 13 and 14). This slight increase in size was most probably due to the attachment of Tf to the liposome surface, which somewhat increases the hydrodynamic diameter of liposomes. The TEM images revealed that the long circulating liposomes were round and of spherical in shape. The zeta potential of Tf conjugated and non-conjugated long circulating liposomes was found to be about −18±3 mV. The Tf conjugation of LCL did not result in any significant change in the zeta potential of the liposomes and was about −17±4 mV.

The stability of paclitaxel liposomal formulations was monitored by changes in particle size and drug retention over a 7 day period during storage at 2-8° C. The colloidal stability of liposomal formulations (size and zeta potential) was excellent with minimal or no change from the initial value. The paclitaxel retention in the liposomes was more than 97% of the label, during 7 day storage period. There were no visible changes to the physical appearance of the formulation or signs of drug precipitation from the lipid bilayers during this 7 day period. Based on free Tf concentration from a typical formulation, about 72% of Tf was coupled to the liposomes. It was found by this method that about 4 μg of the Tf was bound to 1 μM of the total lipid; this corresponds to approximately 5 Tf molecules per 100 nm liposome.

### Example 6

### NIRF Imaging of Tumor Growth in Nude Mice

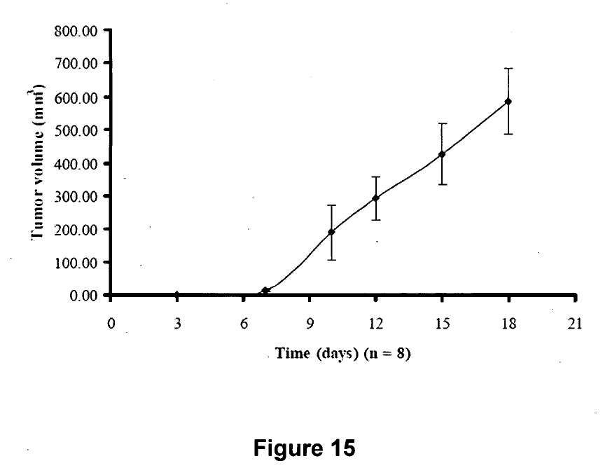

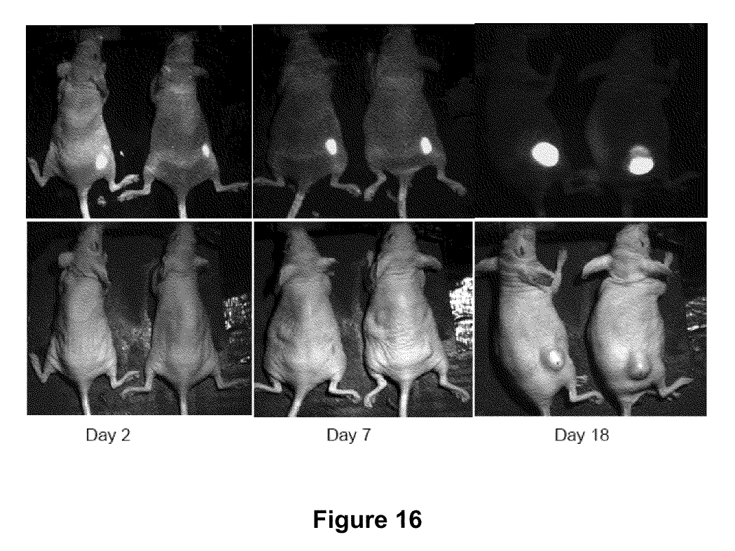

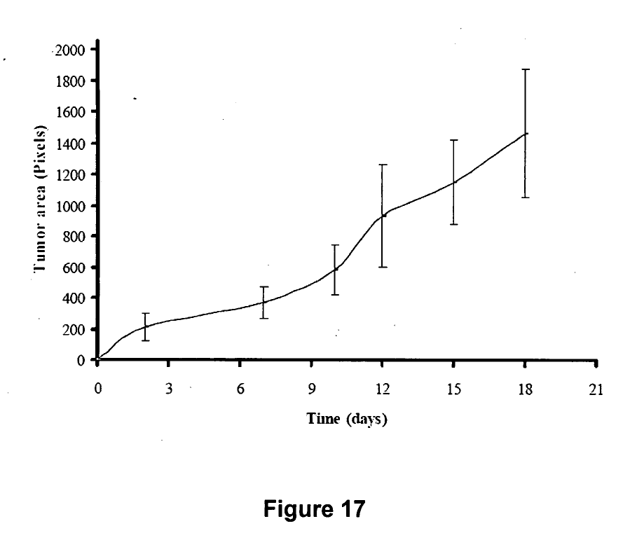

Flank growth of C6 GFP tumor cells was sequentially monitored in a group of 8 mice, using the total light emission over the flank tumor area as an indication of tumor burden. The C6 GFP fluorescence could be detected in mice from day 1 after inoculation of 4,000,000 cells. The tumor distribution was estimated by caliper measurements and correlated with the regions of GFP fluorescence (see FIGS. 15-17). Relative pixel intensity of the tumor increased with time after inoculation (see FIG. 17), and the time taken for flank tumor volume to increase from 30 to 500 mm³ (see FIG. 15) ranged from 5 to about 18 days. Overall, there was good correlation between caliper measurements of the tumors and near infrared fluorescence imaging. However, imaging was helpful in estimating the tumor area more precisely at earlier time points, as caliper measurements could not precisely identify small tumors until approximately day 7 after tumor implantation. In contrast, caliper measurements were found to be more accurate in determining actual tumor volume, when tumors were more than 400 mm³.

### Example 7

### Anti-Tumor Efficacy of Paclitaxel in Liposomes

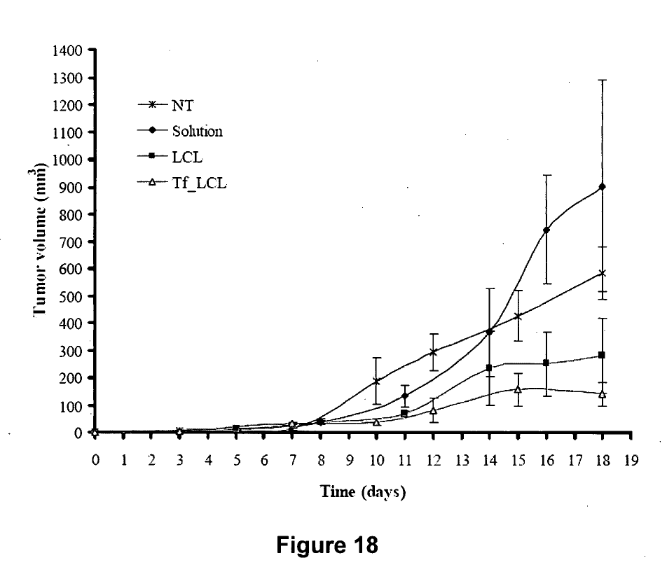

C6 GFP glioma tumors were induced in CD1 nu/nu nude mice by inoculation of GFP-expressing C6 glioma (C6-GFP) cells. The in vivo efficacy of paclitaxel in glioma tumors was evaluated. Tumor areas were measured with METAMORPH® software after taking optical images with a CCD camera with a GFP filter. As shown in FIG. 18, paclitaxel in Tf-LCL formulation resulted in significant tumor growth delay of 7.7 days when compared to 3.3 days with LCL and 0 days with solution formulation. Clinical monitoring and daily weights were similar between groups, thus indicating that no gross deleterious effects of paclitaxel when administered systemically at the tested dose. Thus, transferrin receptor targeted liposomes retained their targeting ability to glioma tumors. Treatment with these liposomal formulations significantly increased the anti-tumoral efficacy of the drug.

Disclosed herein is the discovery that preferential targeting of paclitaxel in to glioma tumors using transferrin-conjugated liposomes results in significant tumor growth delay. Therefore, chemotherapeutic drug delivery targeted to the glioma using transferrin receptor targeted liposomes is a promising new strategy for cancer chemotherapy.

### Materials and Methods for Examples 8-10

Materials: Egg phosphatidylcholine (EPC), hydrogenated soybean phosphatidylcholine (HSPC), 1,2-distearoyl-sn-glycero-3-phosphoethanolamine-N-[PEG(2000)] conjugate (DSPE-PEG), and DSPE-PEG-biotin were from Northern Lipids Inc. (Vancouver, Canada). Cholesterol and biotinylated transferrin were obtained from Sigma (St. Louis, Mo., United States of America) and the DIR fluorescent dye was purchased from Invitrogen Corp. (Carlsbad, Calif., United States of America). These chemicals were used as received. Paclitaxel was purchased from 21 CEC Pharma, East Sussex, United Kingdom. All other chemicals and solvents used were analytical grade.

Liposome preparation: Liposomes were produced by the lipid hydration method followed by extrusion. The liposomes contained egg phosphatidylcholine (EPC), hydrogenated soy phosphatidylcholine (HSPC), cholesterol (75:15:5 molar ratios). Paclitaxel was entrapped in the lipid bilayers due to its lipophilic nature. For preparation of long circulating liposomes (LCL), poly(ethylene glycol-2000)-grafted distearoyl phosphatidyl ethanolamine (DSPE-PEG2000) was incorporated. For preparation of transferrin conjugated liposomes (Tf-LCL) a portion of DSPE-PEG2000 (0.01 mol %) was replaced with DSPE-PEG2000-biotin and then transferrin (Tf) was non-covalently conjugated at the distal end of DSPE-PEG2000-biotin via streptavidin-biotin bond. DIR (lipophilic fluorescent marker) was entrapped in the liposomal bilayers.

Quantification of paclitaxel: A sensitive reverse phase HPLC method was developed and validated to quantitate paclitaxel in liposome formulations. Chromatographic system consisted of a Waters 600 controller, Waters 717 plus auto sampler and a Waters 2996 photodiode array detector. Data were acquired and processed with Waters Millennium 32 software (version 4.0). Chromatographic separation was achieved on a NOVAPAK® C18 reverse phase column (3.9×150 mm) from Waters (Milford, Mass., United States of America). The isocratic mobile phase consisting of acetonitrile and water (55:45, v/v) was pumped at a flow rate of 0.7 mL/min with an injection volume of 20 μL. Paclitaxel (retention time, 3.8 min) was monitored at 230 nm with a photo diode array detector. Prior to HPLC analysis, the formulation samples were treated with methanol for paclitaxel extraction. All analyses were performed in triplicate, and the mean peak area was used to determine the concentration of paclitaxel in the samples. Drug loading was quantified with high-performance liquid chromatography (HPLC). A centrifugal ultrafiltration device (Centricon 100, MWCO 100 kDa; Millipore, Bedford, Mass., United States of America) was used to separate free paclitaxel from the paclitaxel entrapped in the liposomes in the finished product. Total paclitaxel concentration in the liposomes was determined using HPLC after methanol extraction. The percent paclitaxel entrapped into the liposomes was calculated from the free and total paclitaxel in the liposomes.

Liposome size, morphology and zeta-potential: The size analysis of liposomes was performed by dynamic light scattering technique using Malvern zeta sizer-nano particle size analyzer (Malvern Instruments, Malvern, United Kingdom). A 25 μL of this sample was diluted to 1 mL with water for injection for particle size determination. The diluted aqueous sample (1 mL) was added to a 2 mL cuvette and the analysis was performed in triplicate. Average particle size was calculated from the result. The transmission electron microscopic studies were carried out using 3 mm Forman coated copper grid (400 mesh) at 60 KV using negative staining by 2% uranyl acetate at 200,000× magnifications on a JEOL 1200EX transmission electron microscope (TEM).

Quantification of transferrin conjugation: A sensitive gel filtration chromatographic method was developed and validated to quantitate transferrin. The gel filtration chromatographic system consisted of a Waters 600 controller, Waters 717 plus auto sampler and a Waters 2996 photodiode array detector. Data were acquired and processed with Waters Millennium 32 software (version 4.0). Gel filtration chromatographic separation was achieved on a TSK Gel G3000 SWXL (30 cm×7.8 mm, 5 micron) column from Tosoh Bioscience LLC (Montgomeryville, Pa., United States of America). The isocratic mobile phase consisting of 0.5 N phosphate buffered saline (pH 7.2) was pumped at a flow rate of 0.5 mL/min with an injection volume of 30 μL. Transferrin (retention time, 4.8 min) was monitored at 220 nm with a photo diode array detector. All analyses were performed in triplicate, and the mean peak area was used to determine the concentration of transferrin in the samples.

Free Tf was separated from the liposome-entrapped fraction using a Centricon centrifugal filter device (Centricon 100, MWCO 100 kDa; Millipore, Bedford, Mass.). An aliquot of the liposome dispersion (100 μL) was diluted to 1 mL with hydration buffer (phosphate buffered saline pH 7.2). This sample was transferred to the centrifugal filter device. The sample was centrifuged at 5000 rpm for 30 minutes in a fixed-angle centrifuge. Free transferrin in the filtrate was then determined using high performance gel filtration chromatography (HPGFC). Subtraction of free Tf from the amount added gave the amount of liposome-conjugated Tf. Tf estimations were done in triplicate, and the values were reported as mean±standard deviation.

Induction of C6 GFP intracranial glioma tumor xenografts: C6-GFP glioma cells in their exponential growth phase were harvested with 0.25% Trypsin with EDTA for 5 min at 37° C. The cells were centrifuged for 5 min at 1,000 RPM. The pellets were resuspended in sterile phosphate buffered saline (PBS), at a concentration of 100,000,000/mL and placed on ice. Adult CD1 nu/nu mice (weight 25-30 g each; Charles River Laboratories Intl., Inc., Wilmington, Mass., United States of America) were used for all studies and handled in accordance with protocols approved by the Animal Care and Use Committee at the University of Tennessee Health Science Center. Mice were anesthetized with an intraperitoneal injection of ketamine/xylazine at a dosage of 8.7/1.3 mg/100 g body weight. To create the intracranial glioma tumor model, 500,000 C6-GFP glioma cells in 5 μL of phosphate buffered saline were injected into the brain at a depth of 3.0 mm from the surface of skull and 3.0 mm lateral from midline along the bregma suture in the right hemisphere, using a Hamilton syringe and stereotaxic frame. The scalp defect is then closed with cyanoacrylate glue.

NIRF imaging of tumors and tumor localization of DIR labeled liposomes in mice: Fourteen days after tumor cell inoculation, animals were injected retroorbitally with different formulations labeled with a lipophilic dye (DIR). The area and pixel intensity of the dye in the tumor was compared with the background intensity in the surrounding normal tissue using non-invasive optical imaging with a CCD camera with DIR filter at 0, 1, 8, 24 and 48 hours after injection. 48 hours after injection, animals were anesthetized and subjected to transcardiac perfusion first with 20 mL of normal saline and then with same amount of 4% paraformaldehyde to fix the tissue. The brains were incubated in 4% paraformaldehyde. Images of isolated brains were taken with a CCD camera (Princeton Instruments Inc., Trenton, N.J., United States of America) using GFP (excitation: 475 nm and emission: 510 nm) and/or DIR (excitation: 750 nm and emission: 782 nm; Omega Optical, Brattleboro, Vt., United States of America) for visualization of tumor area and DIR dye localization in the brain tissue. Acquired images were processed for measuring the pixel intensity of the DIR fluorescence from the tumors using the METAMORPH® software (version 6.2) for determining the C6 GFP tumor area. Tumor to muscle accumulation ratio of DIR dye labeled formulations was determined to calculate tumor targeting index.

Antitumor efficacy of paclitaxel in liposomes: Five days after tumor cell inoculation, animals were assigned randomly into 3 groups (n=3-4 per group). The groups included no treatment control, paclitaxel in transferrin conjugated long circulating liposomes (Tf-LCL) and paclitaxel in long circulating liposomes (LCL). After 5 days of unrestricted tumor growth, animals were treated with 2 mg/Kg of paclitaxel once a day (for about 9 days) till the end of experiment. The animals were monitored visually on a daily basis and weights were measured on every 3rd day until they lost 10% of their body weight. On the 14th day of tumor implantation, animals were anesthetized and subjected to transcardiac perfusion first with 20 mL of normal saline and then with same amount of 4% paraformaldehyde to fix the tissue. The brains were incubated in 4% paraformaldehyde. Optical images of isolated brains were taken with a CCD camera (Princeton Instruments Inc., Trenton, N.J., United States of America) using a GFP filter. Acquired images were processed using the METAMORPH® software (version 6.2) for determining the C6 GFP tumor area.

Statistics: The in vivo tumor localization and antitumor efficacy data were compared using one-way analysis of variance to determine significant differences among experimental groups. All values of P≦0.05 were considered statistically significant.

### Example 8

### Preparation and Characterization of Transferrin-Conjugated Liposomal Paclitaxel

Paclitaxel incorporation efficiency was determined by subtracting free drug fraction was found to be 98.0±2%. The non-conjugated liposome formulation showed average vesicle sizes of 133±15 nm with a unimodal distribution. The covalent coupling of Tf to the liposome surface led to a slight increase in diameter to about 141±20 nm This slight increase in size most probably due to the attachment of Tf to the liposome surface, which somewhat increases the hydrodynamic diameter of liposomes. The TEM images revealed that the long circulating liposomes were round and of spherical in shape. The zeta potential of Tf conjugated and non-conjugated long circulating liposomes was found to be about −18±3 mV. The Tf conjugation of LCL did not result in any significant change in the zeta potential of the liposomes.

The stability of paclitaxel liposomal formulations was monitored by changes in particle size and drug retention over a 7 day period during storage at 4° C. The colloidal stability of the liposomal formulations (size and zeta potential) was found to be excellent with minimal or no change in from the initial value. The paclitaxel retention in the liposomes was more than 97% of the label, during a 7 day storage period at 2-8° C. There were no visible changes to the physical appearance or signs of drug precipitation from the lipid bilayers during this 7 day period.

The total amount of the liposome attached transferrin for the typical Tf-LCL formulation was determined by using the high performance gel filtration chromatography. Tf dissolved in the mobile phase eluted at ˜4.8-minute retention. Based on free Tf concentration from a typical formulation, about 72% of Tf was coupled to the liposomes. It was found that about 4 μg of the Tf was bound to 1 μM of the total lipid; this corresponds to approx. 5 Tf molecules per 100 nm liposome.

### Example 9

### NIRF Imaging of Tumors and Tumor Localization of DIR-Labeled Liposomes in Mice

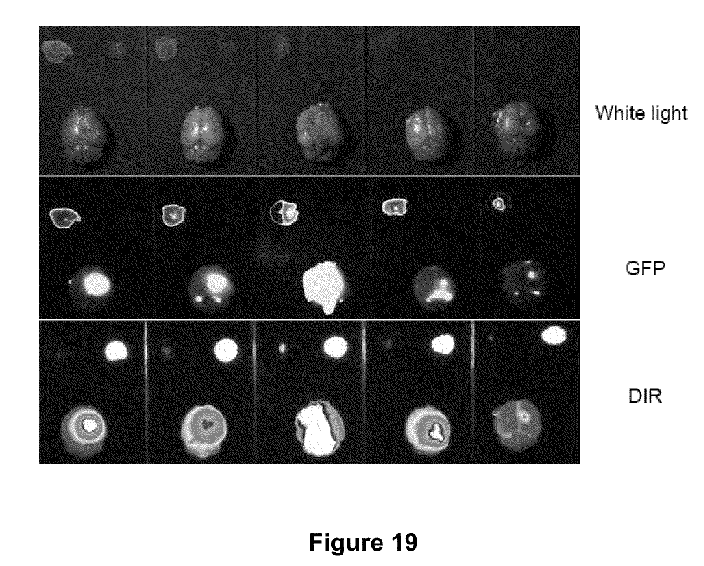

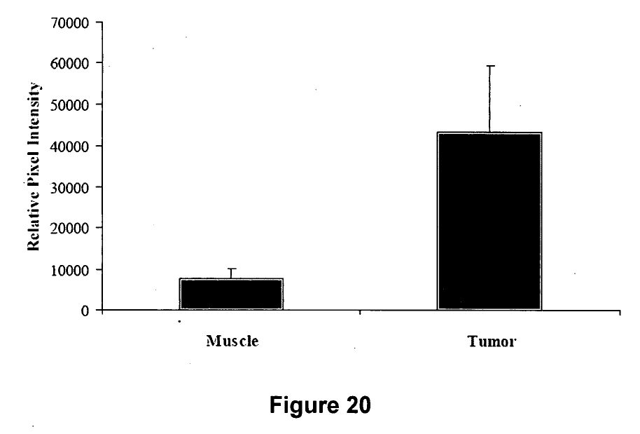

An enhanced permeation and retention (EPR) effect has been demonstrated for nano carriers in tumor targeting (Papisov, 1998; Shan et al., 2006). The tumor accumulation and the tumor-to-muscle accumulation ratio of liposomal formulation using a non-invasive NIR fluorescence imaging method was investigated. The data showed that the LCL and Tf-LCL selectively accumulated in C6 intracranial glioma tumors (see FIG. 19). The Tf-LCL formulation accumulated in the C6 glioma tumors more efficiently as compared to muscle tissue (see FIG. 20). The tumor targeting index for Tf-LCL was found to be 6.15±1.47.

### Example 10

### Anti-Tumor Efficacy of Paclitaxel in Liposomes

To study the effect of paclitaxel on C6 GFP glioma in vivo, tumors were induced in CD1 nu/nu nude mice by intracranial inoculation of GFP-expressing C6 glioma (C6-GFP). After 5 days of tumor cell inoculation, animals were assigned randomly to 3 groups (n=3-4 per group). These groups were paclitaxel Tf-LCL, LCL, and no treatment control. Drug was administered once a day via retro orbital injection for 9 subsequent days with 2 mg/Kg in Tf-LCL or LCL formulation. The animals were then sacrificed on the 9^{th} day of treatment and the brains were isolated.

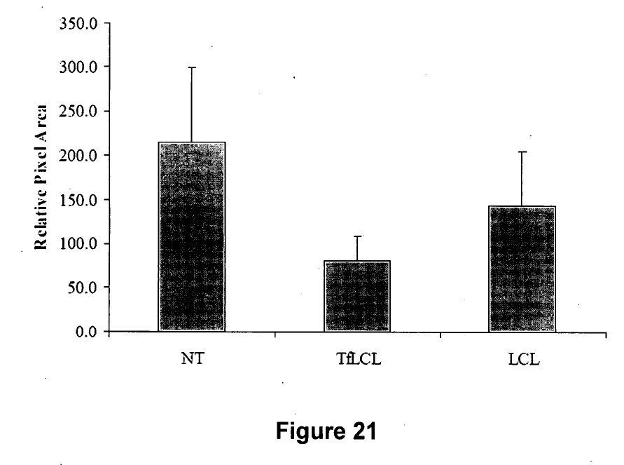

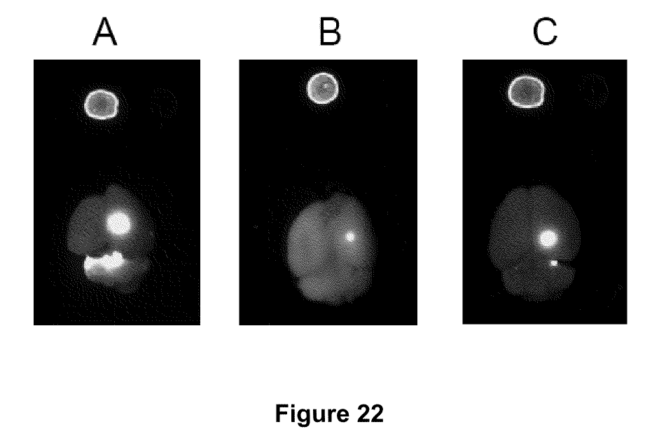

Tumor areas were measured METAMORPH® software after taking optical images with a CCD camera with GFP filter. As shown in FIG. 21, paclitaxel in Tf-LCL formulation resulted in significant tumor reduction when compared to no treatment tumor control (p=0.038). No statistical significant tumor reduction was observed with long circulating liposomes (p=0.188). FIGS. 22A and 22B show representative photomicrographs of the average tumor area in animals treated with paclitaxel in Tf-LCL formulation compared with paclitaxel in LCL and no treatment control group. Clinical monitoring and daily weights were similar between groups indicating no gross deleterious effects of paclitaxel when administered systemically at the tested dose. Paclitaxel encapsulation in Tf-LCL formulation group was more active than the non-treated, and paclitaxel in long circulating liposome groups for the treatment of the intracranial glioma tumor at the 2 mg/Kg dose. Whole body NIRF imaging of mice implanted with C6-GFP intracranial glioma revealed marked selective intracranial glioma localization with Tf-LCL formulation.

These studies provide evidence that paclitaxel delivered in Tf-LCL accumulate effectively with relative selectivity in tumor areas, improving the overall anti-tumor efficacy in intracranial gliomas over a non targeted liposomal system.

### Materials and Methods for Example 11

Materials: Egg phosphatidylcholine (EPC), hydrogenated soybean phosphatidylcholine (HSPC), and 1,2-distearoyl-sn-glycero-3-phosphoethanolamine-N-[PEG(2000)] conjugate (DSPE-PEG) were from Northern Lipids Inc., Vancouver, Canada. Cholesterol, biotinylated transferrin, sucrose, and trehalose were obtained from Sigma (St. Louis, Mo., United States of America). These chemicals were used as received. Paclitaxel was purchased from 21CEC Pharma, East Sussex, United Kingdom. All other chemicals and solvents used were of analytical grade.

Liposome preparation: Liposomes were produced by the lipid hydration method followed by extrusion. The liposomes contained egg phosphatidylcholine (EPC), hydrogenated soy phosphatidylcholine (HSPC), cholesterol (75:15:5 molar ratios). The paclitaxel (log P is 3.96) was entrapped in the lipid bilayers due to its lipophilic nature. For preparation of long circulating liposomes (LCL), poly(ethylene glycol-2000)-grafted distearoyl phosphatidyl ethanolamine (DSPE-PEG2000) was incorporated. For preparation of Tf-LCL a part of DSPE-PEG2000 (0.01 mol %) was replaced with DSPE-PEG2000-biotin and then transferrin (Tf) was non-covalently conjugated at the distal end of DSPEPEG2000-biotin via streptavidin-biotin bond.

Lyophilization of liposomes: For cryoprotection of liposomes during lyophilization, 15% sucrose or trehalose was added to freshly prepared liposome dispersions and distributed into 5 mL freeze-dry vials (Wheaton Glass Company, Millville, N.J., United States of America) in 1 mL aliquots. The rubber freeze-dry closures (type V9172-FM 257; Helvoet Pharma, Alken, Belgium) were used. Vials, partially stoppered with freeze-dry closures, were loaded into the Virtis genesis freeze-dryer and frozen slowly to −45° C. at 1° C. per min. Freezing was continued for 5 hours. At the end of the freezing step, chamber pressure was brought to 110-120 m torr and the shelf temperature was maintained at −35° C. for 10 hours, followed by drying at shelf temperature of −20° C. for 5 hours and at 0° C. for 5 hours (primary drying). The secondary drying was carried out at 50-60 m torr chamber pressure at 10° C. for 14 hours and at 20° C. for 10 hours. The condenser temperature ranged between −55 and −60° C. At the end of the freeze-drying process the chamber was backfilled with nitrogen to maintain a nonreactive gaseous headspace. The vials were closed with rubber closures were unloaded from the chamber followed by crimping with aluminum seals.

Liposome size, morphology and zeta-potential: Lyophilized samples were reconstituted (rehydrated) to their original volume (1 mL) with sterile water for injection. A 25 μL of this sample was diluted to 1 mL with water for injection for particle size determination. The average size and polydispersity index were determined at 25° C. by dynamic light scattering method with Malvern Zetasizer Nano, using the dispersion technology software version 4.10 (Malvern Ltd, Malvern, United Kingdom). Measurements were performed on three independently prepared samples for each formulation. The same sample was used for zeta potential analysis of liposomes with Malvern Zetasizer Nano, using the dispersion technology software version 4.10 (Malvern Ltd, Malvern, United Kingdom).

Estimation of drug entrapment in liposome formulation: A sensitive reverse phase HPLC method was developed and validated to quantitate paclitaxel in liposome formulations. Chromatographic system consisted of a Waters 600 controller, Waters 717 plus auto sampler and a Waters 2996 photodiode array detector. Data were acquired and processed with Waters Millennium 32 software (version 4.0). Chromatographic separation was achieved on a NOVAPAK® C18 reverse phase column (3.9×150 mm) from Waters (Milford, Mass., United States of America). The isocratic mobile phase consisting of acetonitrile and water (55:45, v/v) was pumped at a flow rate of 0.7 mL/min with an injection volume of 20 μL. Paclitaxel (retention time, 3.8 min) was monitored at 230 nm with a photo diode array detector. Prior to HPLC analysis, the formulation samples were treated with methanol for paclitaxel extraction. All analyses were performed in triplicate, and the mean peak area was used to determine the concentration of paclitaxel in the samples. The total and free paclitaxel in the liposome formulations before and after lyophilization and reconstitution were determined using an HPLC method of analysis. The drug loading was quantified via high-performance liquid chromatography (HPLC). Briefly, a centrifugal ultrafiltration device (Centricon 100, MWCO 100 kDa; Millipore, Bedford, Mass., United States of America) was used to separate free paclitaxel from the paclitaxel entrapped in the liposomes. Free and total paclitaxel concentration in the liposomes was determined using HPLC after methanol extraction. Percent paclitaxel entrapped in the liposomes was calculated from the free and total paclitaxel in the liposomes.

### Example 11

### Development of a Lyophilized Targeted Liposome Delivery System for Paclitaxel

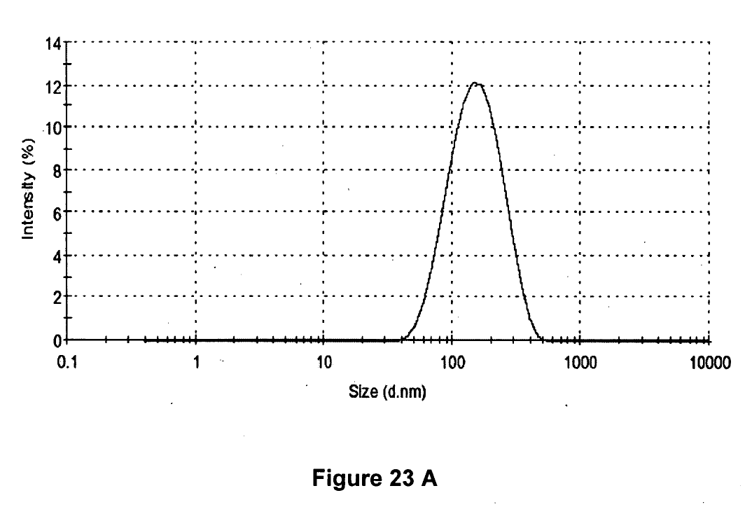

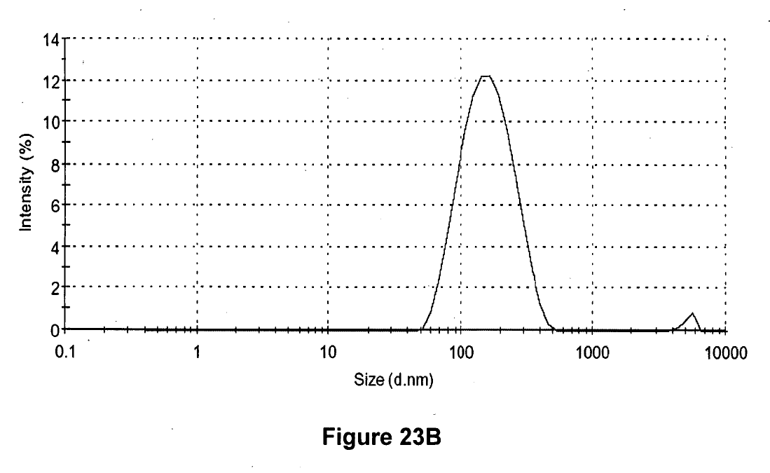

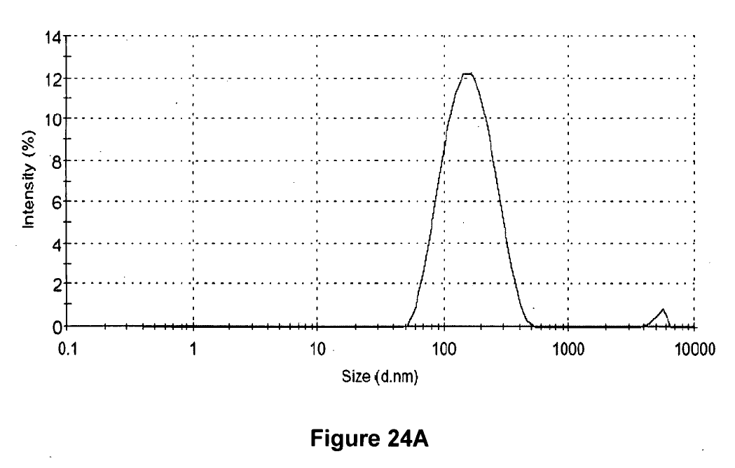

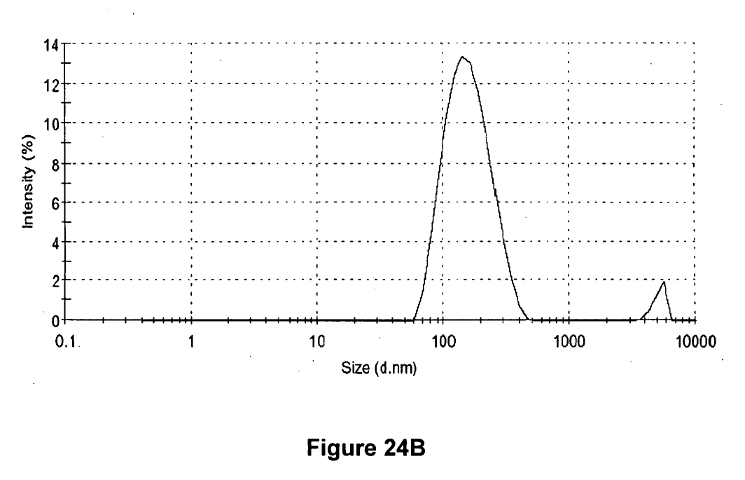

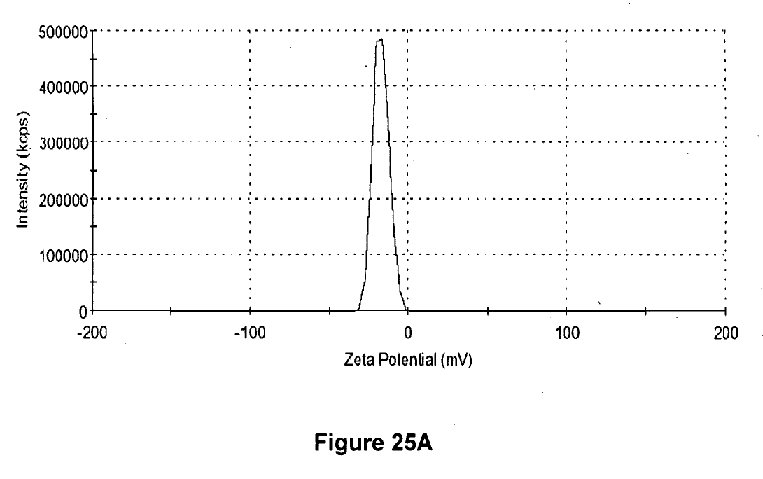

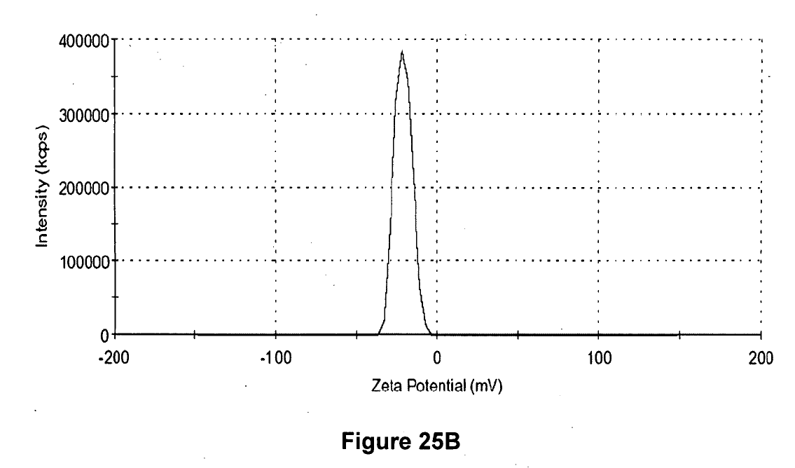

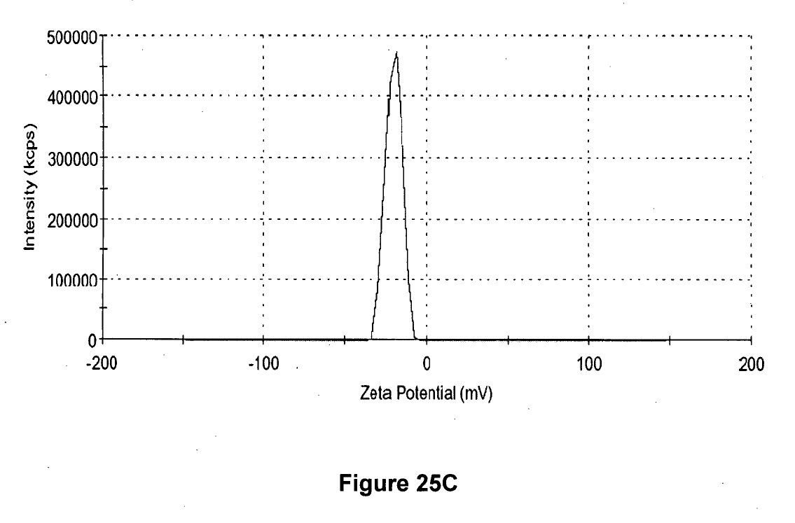

A formulation containing 15% (w/v) extra-liposomal sucrose or 15% (w/v) extraliposomal trehalose were able to maintain the particle size distribution (PSD) of targeted liposomes close to initial after the lyophilization and rehydration (see FIGS. 23A and 23B). These formulations were also maintained drug loading after lyophilization and reconstitution. Lyoprotective effects of sucrose and trehalose are compared in FIGS. 24A and 24B. Zeta potential of liposomes before lyophilization was about −17.6 mV. The average zeta potential of liposomes with 15% sucrose or trehalose as cryoprotectant after lyophilization and reconstitution were about −20 mV (see FIGS. 25A-25C). As can bee seen from these Figures, both of these disaccharides were able to maintain the initial monodisperse size distribution and zeta potential of the targeted liposomes after freeze-drying and reconstitution. But the sucrose was slightly better on maintaining the original PSD and zeta potential of these targeted liposomes than trehalose. Average size and poly dispersity index (PDI) after freeze-drying and reconstitution for trehalose formulation were 161 nm and 0.271, respectively (see FIGS. 24A and 24B). Where as those numbers for the sucrose formulation were 154 nm and 0.25, respectively (see FIGS. 24A and 24B). The size distribution of targeted liposomes before freeze-drying (average size of 137 nm and PDI of 0.176; see FIGS. 23A and 23B) was closely preserved after freeze-drying and reconstitution when sucrose was used as lyoprotectant. Similar results were reported in the literature for some other liposome systems. Sucrose was found to be more effective in maintaining size distribution of mitoxantrone liposomes after lyophilization and reconstitution (Ugwu et al., 2005).

There was no significant change in the size and zeta potential of the liposomes after lyophilization indicated the cryoprotection of the liposomes by the sucrose. In the absence of cryoprotectants, aggregation and fusion of liposomes is expected when lyophilized without cryoprotection. Because liposome bilayer formation itself requires presence of water, removal of this water through freeze-drying should be expected to cause irreversible damage to these structures (Winterhalter & Lasic, 1993). But this is not the case with cryoprotectants. Several reports describe this process of liposome aggregation on lyophilization (Van Bommel & Crommelin, 1984; Van Winden et al., 1997; Van Winden & Crommelin, 1999; Ugwu et al., 2005; Zhang et al., 2005). The most successful lyoprotectants were the non-reducing disaccharides sucrose and trehalose (Van Bommel & Crommelin, 1984; Van Winden & Crommelin, 1999). Based on this information a 15% concentration of sucrose or trehalose was used in the disclosed formulations to protect the targeted liposomes during freeze-drying process.

A lyophilized formulation was developed to increase storage stability of the targeted liposome formulation. Sucrose was observed to be a better lyoprotectant for this formulation and process when compared to trehalose.

### Discussion of the Examples

Liposome drug delivery systems preferentially aimed at glioma tumors are disclosed herein. Liposomes comprising egg phosphatidylcholine (EPC), hydrogenated soybean phosphatidylcholine (HSPC), cholesterol, distearoyl phosphoethanolamine-PEG-2000 conjugate (DSPE-PEG) and DSPE-PEG-maleimide were prepared by the lipid film hydration and extrusion process. Transferrin (Tf) molecules, which can bind to transferrin receptors that are overexpressed on blood brain barrier and glioma tumor vasculature, were coupled to the distal end of polyethylene glycol coated long circulating liposomes.

The liposome delivery systems were characterized in terms of size, lamellarity, ligand density, and drug loading properties. The effect of lipid concentration and type in the formulation on paclitaxel loading in the liposomes was studied. Functional properties of the delivery systems were evaluated for i) in vivo blood circulation time using blood sampling method and also using a novel intravital microscopic method; ii) selective tumor localization in both flank and intracranial glioma models; and iii) anti-tumor efficacy in mouse flank and intracranial glioma tumors.

Further, in order to improve physical and chemical stability of the delivery system and hence enhance its shelf life, lyophilized formulations and processes were also prepared.

Light scattering and electron microscopic observations of the formulations revealed the presence of small unilamellar liposomes of about 133 nm in diameter. High performance gel filtration chromatography determinations of ligand coupling to the liposome surface indicated that about 72% of the transferrins were conjugated with biotin groups on the liposome surface. A representative liposome formulation with 100 mM lipid concentration, 1:33 drug-to-lipid ratio, 5 mol % cholesterol, 5 mol % DSPE-PEG, and 0.01 mol % DSPE-PEG-biotin content yielded 1.3±0.2 mg/mL liposomal paclitaxel with 97.2±3% of the drug being entrapped in the liposomes. These liposomes released no significant amount of the entrapped drug over 72 hours at 37° C.

Targeted liposomes showed significantly higher rate and extent of tumor accumulation in glioma flank tumors in vivo compared to non-targeted liposomes. Targeted liposomes also possessed long circulating properties with a T_{1/2} of about 9 hours in mice. This increased circulation longevity, attributed to steric stabilization effects of PEG, enhanced target accumulation.

Near infrared fluorescence imaging demonstrated that these liposomes accumulated selectively in flank tumors with tumor targeting index of 10.59±1.08. Paclitaxel incorporated into the targeted liposomes delayed tumor growth by 7.7 days in 5 doses of 2 mg/Kg body weight. However, no significant tumor growth retardation was observed when paclitaxel was administered in free form (CREMOPHOR EL® solubilized form) at similar dose.

An objective of the investigations disclosed herein was to develop a targeted liposome delivery system for the paclitaxel, to selectively deliver this drug to glioma brain tumors. Towards this goal, first, a targeted liposome delivery system targeting transferrin receptors highly expressed on gliomas was designed. Then, a formulation and process for preparation of this targeted liposome delivery system was developed. The functional properties of the delivery system were evaluated both in vitro and in vivo. The delivery system was characterized and the formulation was optimized to achieve maximum in vivo blood circulation half-life and high tumor localization. Finally, to enhance the storage stability of the delivery system, a lyophilized formulation and process were developed.

The elements of the targeted delivery system were chosen to satisfy a number of desirable characteristics such as high drug loading, stable encapsulation, good physical and chemical stability during shelf life, long circulation half-life in vivo, high tumor targeting potential, and efficient target recognition and binding. An exemplary targeted liposome delivery system comprises five different lipid components (EPC, HSPC, cholesterol, DSPE-PEG, DSPE-PEG-biotin) and a targeting ligand, transferrin. The targeting ligand transferrin was coupled to the distal ends of PEG chains. This specific way of ligand coupling to the liposome carrier was used to optimize ligand coupling to the liposome carrier as well as their interaction with the intended biological targets (transferrin receptors on glioma tumors).

The extrusion process developed for the preparation of these liposome carriers is robust. There was a great control of liposome size and size distribution in the extrusion process used. The process can be scaled up easily. A long blood circulation half-life in rats (9 hours) for fluorescent labeled targeted formulations was achieved. The sterically stabilized liposomes prepared using 5% PEG and about 100 nm in size showed a pronounced increase in the blood residence time (17 hours) with a significant decrease in uptake by RES when compared with conventional liposomes. The rate extent of tumor localization of the transferrin receptor targeted fluorescent liposome was found to be significantly high compared to non targeted liposomes and solution formulations.

Anti-tumor efficacy evaluation in intracranial and flank tumor models indicated that targeting paclitaxel to glioma brain tumors using this liposome delivery system was significantly more effective than free paclitaxel administered in a micellar solubilized dosage form or non targeted liposomes. This targeted delivery system also reduced the dose required for the therapeutic efficacy. Further unwanted toxic effects in other normal tissues were not observed. The significant tumor growth delay was observed. Using a multiple dosing regimen, such as repeated administration of radiation and targeted liposomes containing paclitaxel, would likely further delay the tumor growth.

Finally, a successful lyophilization formulation and process was developed to enhance the storage stability of the targeted liposome delivery system. A formulation containing 15% (w/v) extra-liposomal sucrose was able to maintain the particle size distribution and drug loading of the targeted liposomes close to initial after freeze-drying and rehydration.

Summarily, disclosed herein are stable and effective targeted liposome delivery systems for paclitaxel to take this drug selectively to glioma brain tumors. These targeted delivery systems could potentially increase the anti-cancer activity as well as the therapeutic index of the drug compared to existing solution dosage forms.

### REFERENCES

The references listed below as well as all references cited in the specification, including patents, patent applications, journal articles, and all database entries (e.g., GENBANK® and/or National Center for Biotechnology Information (NCBI) Accession numbers, including all annotations presented in the various databases associated with the disclosed sequences), are incorporated herein by reference to the extent that they supplement, explain, provide a background for, or teach methodology, techniques, and/or compositions employed herein.
Allen (1997) Journal of Liposome Research 7:315-329.
Allen (2002) Nature Reviews Cancer 2:750-763.
Allen (2004) Proceedings—2004 International Conference on MEMS, NANO and Smart Systems, ICMENS 2004.
Allen et al. (2002) Cellular and Molecular Biology Letters 7:889-894.
Berkow et al. (1997) The Merck Manual of Medical Information, Home ed. Merck Research Laboratories, Whitehouse Station, N.J., United States of America.
Creighton (1983) Proteins: Structures and Molecular Principles, W.H. Freeman & Co., New York, N.Y., United States of America.
Crowe (2007) Advances in Experimental Medicine and Biology 594:143-158.
Crowe & Crowe (1988) Biochimica et Biophysica Acta (BBA)—Biomembranes 939:327-334.
Crowe et al. (1987) Biochemical Journal 242:1-10.
Crowe et al. (1997) Cryobiology 35:20-30.
Crowe et al. (1998) Annual Review of Physiology 60:73-103.
Cui et al. (2006) International Journal of Pharmaceutics 312:131-136.
Dorr et al. (1997) Cancer Chemotherapy Handbook, 2d edition, Appleton & Lange, Stamford, Conn., United States of America.
Drummond et al. (1999) Pharmacological Reviews 51:691-743.
Duch et al. (1998) Toxicology Letters 100-101:255-263.
Ebadi (1998) CRC Desk Reference of Clinical Pharmacology. CRC Press, Boca Raton, Fla., United States of America.
Freireich et al. (1966) Cancer Chemotherapy Reports 50:219-244.
Gaber et al. (2004) Brain Research Protocols 13:1-10.
Gabizon et al. (1999) Bioconjugate Chemistry 10:289-298.
Glavas-Dodov et al. (2005) International Journal of Pharmaceutics 291:79-86.
Golub & Gren (1991) Immunology—A Synthesis, 2nd Edition, Sinauer Associates, Sunderland, Mass., United States of America.
Goodman et al. (1996) Goodman & Gilman's the Pharmacological Basis of Therapeutics, 9th ed. McGraw-Hill Health Professions Division, New York, N.Y., United States of America.
Green (1964) Biochemical Journal 92:16C-17C.
Green (1990) Methods in Enzymology 184:51-67.
Green & Joynson (1970) Biochemical Journal 118:71-72.
Hansen et al. (1995) Biochimica et Biophysica Acta—Biomembranes 1239:133-144.
Harashima et al. (1994) Pharmaceutical Research 11:402-406.
Harasym et al. (1998) Advanced Drug Delivery Reviews 32:99-118.
Henikoff & Henikoff (2000) Advanced Protein Chemistry 54:73-97.
Henley et al. (2007) Cancer Chemotherapy and Pharmacology 59:235-249.
Hennenfent & Govindan (2006) Annals of Oncology 17:735-749.
Ishida et al. (1999) FEBS Letters 460:129-133.
Jovanovic et al. (2006) European Journal of Pharmaceutical Sciences 27:336-345.
Katzung (2001) Basic & Clinical Pharmacology, 8th ed. Lange Medical Books/McGraw-Hill Medical Pub. Division, New York, N.Y., United States of America.
Kyte et al. (1982) Journal of Molecular Biology 157:105-131.
Lasic (1994) Angewandte Chemie (International Edition in English) 33:1685-1698.
Lasic (1997) Journal of Controlled Release 48:203-222.
Lasic et al. (1991) Biochimica et Biophysica Acta—Biomembranes 1070:187-192.
Leibel & Phillips (1998) Textbook of Radiation Oncology, Saunders, Philadelphia, United States of America.
Loughrey et al. (1987) Biochimica et Biophysica Acta—Biomembranes 901:157-160.
Markman et al. (1994) Annals of Oncology: Official Journal of the European Society for Medical Oncology/ESMO 5 Suppl 6:S55-58.
Martin et al. (1981) Biochemistry 20:4229-4238.
Papisov (1998) Advanced Drug Delivery Reviews 32:119-138.
Partridge (1999a) Journal of NeuroVirology 5:556-569.
Pardridge (1999b) Advanced Drug Delivery Reviews 36:299-321.
Pardridge (1999c) Pharmaceutical Science and Technology Today 2:49-59.
Pardridge (2002a) Nature Reviews Drug Discovery 1:131-139.
Pardridge (2002b) Archives of Neurology 59:35-40.
PCT International Patent Application Publication No. WO 93/25521.
Peer et al. (2003) Biochimica et Biophysica Acta—Biomembranes 1612:76-82.
Qian (2002) Pharmacological Reviews 54:561-587.
Remington et al. (1975) Remington's Pharmaceutical Sciences, 15th ed. Mack Pub. Co., Easton, Pa., United States of America.
Rowinsky & Donehower (1993) Seminars in Oncology 20:16-25.
Rowinsky et al. (1993) Cancer Surveys 17:283-304.
Rowinsky et al. (1994) Annals of Oncology: Official Journal of the European Society for Medical Oncology/ESMO 5 Suppl 6:S7-16.
Sakahara & Saga (1999) Advanced Drug Delivery Reviews 37:89-101.
Schnyder et al. (2004) Biochemical Journal 377:61-67.
Shan et al. (2006) Cancer Chemotherapy and Pharmacology 58:245-255.
Sharma & Sharma (1997) International Journal of Pharmaceutics 154:123-140.
Sharma & Straubinger (1994) Pharmaceutical Research 11:889-896.
Slavin et al. (2007) Cardiology in Review 15:1-12.
Speight et al. (1997) Avery's Drug Treatment: A Guide to the Properties Choice, Therapeutic Use and Economic Value of Drugs in Disease Management, 4th ed. Adis International, Philadelphia, Pa., United States of America.
Straubinger et al. (1993) Journal of the National Cancer Institute. Monographs:69-78.
Sun & Eikelboom (2007) New England Journal of Medicine 356:1071.
Sun et al. (1996) Biophysical Journal 70:1769-1776.
Tarr & Yalkowsky (1987) Journal of Parenteral Science and Technology 41:31-33.
Thompson et al. (1997) Nucleic Acids Research 24:4876-4882.
Tirosh et al. (1998) Biophysical Journal 74:1371-1379.
Torchilin (2004) Cellular and Molecular Life Sciences 61:2549-2559.
Torchilin (2005a) Advanced Drug Delivery Reviews 57:95-109.
Torchilin (2005b) Nature Reviews Drug Discovery 4:145-160.
Ugwu et al. (2005) Drug Development and Industrial Pharmacy 31:223-229.
U.S. Pat. Nos. 4,554,101; 5,234,933; 5,326,902; 6,180,082.
Van Bommel & Crommelin (1984) International Journal of Pharmaceutics 22:299-310.
Van Winden & Crommelin (1999) Journal of Controlled Release 58:69-86.
Van Winden et al. (1997) Pharmaceutical Research 14:1151-1160.
Winterhalter & Lasic (1993) Chemistry and Physics of Lipids 64:35-43.
Yuan et al. (1995) Cancer Research 55:3752-3756.
Zhang et al. (2005) European Journal of Pharmaceutics and Biopharmaceutics 59:177-187.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for inhibiting undesirable proliferation of a tumor cell or a tumor in a subject, the method comprising administering to the subject a composition comprising an effective amount of an active agent, wherein:
(i) the active agent is entrapped by one or more liposomes;
(ii) the active agent has activity in inhibiting undesirable proliferation of the tumor cell or the tumor; and
(iii) the one or more liposomes comprise one or more targeting agents that preferentially or specifically bind to a binding molecule expressed by the tumor cell or the tumor, present on the tumor cell or the tumor, present in the tumor cell or the tumor, or combinations thereof,
whereby the liposomes deliver the active agent to the tumor cell or the tumor.

2. The method of claim 1, wherein the tumor cell or the tumor is a glioma cell or a glioma.

3. The method of claim 1, wherein one or more of the one or more liposomes are long-circulating liposomes.

4. The method of claim 3, wherein the long-circulating liposomes comprise about 1-7% of DSPE-PEG2000 in total lipids.

5. The method of claim 1, wherein the one or more liposomes are less than about 300 nm in diameter.

6. The method of claim 5, wherein the one or more liposomes are about 100 nm in diameter.

7. The method of claim 1, wherein the active agent is selected from the group consisting of docetaxel, camptothecin, carmustine/BCNU, lomustine/CCNU, vincristine, vinblastine, doxorubicin, and paclitaxel.

8. The method of claim 7, wherein the active agent is paclitaxel.

9. The method of claim 8, wherein the paclitaxel is contained within an interior space of the one or more liposomes, attached to an exterior surface of the one or more liposomes, present within a bilayer of the one or more liposomes, or a combination thereof.

10. The method of claim 1, wherein the one or more targeting agents comprises a transferrin polypeptide or a fragment or derivative thereof that preferentially or specifically binds to a binding molecule present on or in the tumor cell or the tumor.

11. The method of claim 10, wherein the binding molecule comprises a transferrin receptor that specifically binds to the transferrin polypeptide or the fragment or derivative thereof.

12. The method of claim 11, wherein the transferrin polypeptide is expressed at substantially greater levels on or in the tumor cell or the tumor than in normal tissue surrounding the tumor cell or the tumor in the subject.

13. The method of claim 1, wherein the one or more liposomes are suspended in a pharmaceutically acceptable carrier.

14. The method of claim 1, wherein the administering comprises intravenously injecting the composition into the subject.

15. The method of claim 1, wherein the administering comprising delivering the composition intracranially to the subject.

16. The method of claim 1, wherein the inhibiting comprises reducing the size of the tumor, reducing tumor load, reducing tumor growth rate, or a combination thereof in the subject.

17. A targeted liposome delivery system for treating a tumor cell or a tumor, the targeted liposome delivery system comprising:
(i) a plurality of liposomes, each liposome comprising an interior space, a lipid bilayer, and an exterior surface;
(ii) an active agent present within the interior space of the liposome, present within the lipid bilayer of the liposome, attached to the exterior surface of the liposome, or a combination thereof; and
(iii) a targeting agent that preferentially or specifically binds to a binding molecule present on or in the tumor cell or the tumor attached to the liposome.

18. The targeted liposome delivery system of claim 17, wherein the plurality of liposomes are lyophilized for enhancement of long-term stability.

19. The targeted liposome delivery system of claim 17, wherein the plurality of liposomes are produced by a lipid hydration method followed by extrusion.

20. The targeted liposome delivery system of claim 17, wherein the liposome comprises one or more lipids selected from the group consisting of egg phosphatidylcholine (EPC), hydrogenated soy phosphatidylcholine (HS PC), cholesterol, 1,2-distearoyl-glycero-3-phosphoethanolamine-N-[PEG(2000)] conjugate (DSPE-PEG), DSPE-PEG-Maleimide, and DSPE-PEG-biotin.

21. The targeted liposome delivery system of claim 17, wherein the targeting agent is attached to the exterior surface of the liposome through an avidin-biotin bond or a thioether bond.

22. The targeted liposome delivery system of claim 17, wherein the plurality of liposomes are suspended in a pharmaceutically acceptable carrier.

23. The targeted liposome delivery system of claim 17, wherein the active agent is selected from the group consisting of docetaxel, camptothecin, carmustine/BCNU, lomustine/CCNU, vincristine, vinblastine, doxorubicin, and paclitaxel

24. The targeted liposome delivery system of claim 23, wherein the active agent is paclitaxel.

25. The targeted liposome delivery system of claim 17, wherein the targeting agent comprises a transferrin polypeptide or a fragment or derivative thereof, and the binding molecule comprises a transferrin receptor that preferentially or specifically binds to the transferrin polypeptide or the fragment or derivative thereof.

26. An anti-tumor therapeutic composition for treating a tumor comprising a suspension of liposomes in a pharmaceutically acceptable carrier, wherein the liposomes comprise:
(a) an interior space, a lipid bilayer, and an exterior surface;
(b) an active agent contained within the interior space of the liposome, present within the lipid bilayer of the liposome, attached to the exterior surface of the liposome, or a combination thereof; and
(c) one or more transferrin polypeptides or fragments or derivatives thereof attached to the liposome.

27. The anti-tumor therapeutic composition of claim 26, wherein the active agent is selected from the group consisting of docetaxel, camptothecin, carmustine/BCNU, lomustine/CCNU, vincristine, vinblastine, doxorubicin, and paclitaxel.

28. The anti-tumor therapeutic composition of claim 27, wherein the active agent is paclitaxel.
